(19) United States
(12) Patent Application Publication
(10) Pub. No.: US 2004/0091855 A1
(43) Pub. Date: May 13, 2004

(54) Method for production of antivirals by use of HIV-derived HR1 peptides, and trimers formed therefrom

(76) Inventors: John Dwyer, Chapel Hill, NC (US); Mary K. Delmedico, Raleigh, NC (US)

Correspondence Address: M. Bud Nelson, Trimeris, Inc., Suite 300, 3518 Westgate Drive, Durham, NC, 27707, US

(21) Appl. No.: 10/671,316

(22) Filed: Sep. 24, 2003

Related U.S. Application Data
(60) Provisional application No. 60/414,515, filed on Sep. 27, 2002.

(51) Int. Cl. C12Q 1/70; C07K 14/00

(52) U.S. Cl. 435/5; 530/350; 530/324

(57) ABSTRACT

Provided is a method for identifying or producing a molecule having antiviral activity against HIV. More particularly, provided is a method for identifying or producing a molecule that can inhibit the binding between HR1 and HR2 regions of HIV gp41, wherein complex formation is observed in vitro between a trimer with HR2 peptide in the presence of the molecule, and detected in vitro is the ability of the molecule to inhibit complex formation as an indicator of the antiviral activity of the molecule. The trimer is comprised of synthetic peptide comprising an amino acid sequence derived from the HR1 region of HIV-1 gp41 and further comprising one or more amino acid substitutions in a hydrophobic domain of the HR1 region of HIV which enable the synthetic peptide to self-assemble in solution into trimers.

This application claims the benefit of the U.S. Provisional Application 60/414,515 filed on 27 Sep. 2002.

### FIELD OF THE INVENTION

The present invention relates to synthetic peptides derived from the HR1 region of gp41, and particularly trimers formed therefrom, and their use for production of other antiviral agents having activity against HIV (Human Immunodeficiency Virus). More particularly, the present invention comprises a method for producing antivirals that can inhibit transmision of HIV, by using trimers formed from HR1-derived peptides which contain one or more site-specific amino acid substitutions (as compared to the native sequence) such that the peptide self-associates in solution to substantially a trimeric form.

### BACKGROUND OF THE INVENTION

It is now well known that cells can be infected by HIV through a process by which fusion occurs between the cellular membrane and the viral membrane. The generally accepted model of this process is that the viral envelope glycoprotein complex (gp120/gp41) interacts with cell surface receptors on the membranes of the target cells. Following binding of gp120 to cellular receptors (e.g., CD4 in combination with a chemokine co-receptor such as CCR-5 or CXCR-4), induced is a conformational change in the gp120/gp41 complex that allows gp41 to insert into the membrane of the target cell and mediate membrane fusion.

The amino acid sequence of gp41, and its variation among different strains of HIV, is well known. FIG. 1 is a schematic representation of the generally accepted functional domains of gp41 (note the amino acid sequence numbers may vary slightly depending on the HIV strain). The fusion peptide (fusogenic domain) is believed to be involved in insertion into and disruption of the target cell membrane. The transmembrane domain, containing the transmembrane anchor sequence, is located at the C-terminal end of the protein. Between the fusion peptide and transmembrane anchor are two distinct regions, known as heptad repeat (HR) regions, each region having a plurality of heptads. The HR1 region, nearer to the N-terminal end of the protein than the HR2 region, has been generally described as comprising the amino acid sequence having the sequence of SEQ ID NO:1. However, due to naturally occurring polymorphisms, the amino acid sequence (and also numbering of residues) of the HR1 region of HIV gp41 may vary, depending on the viral strain from which the amino acid sequence was deduced. The amino acid sequence comprising the HR1 region is one of the most highly conserved regions in the HIV-1 envelope protein (Shu et al., 1999, Biochemistry, 38:5378-5385). The other region, HR2, is also depicted in FIG. 1, and can vary with polymorphisms thereof. The HR regions are known to comprise a plurality of 7 amino acid residue stretches or “heptads” (the 7 amino acids in each heptad designated “a” through “g”), wherein the amino acids in the “a” position and “d” position are generally hydrophobic. Also present in each HR region is one or more leucine zipper-like motifs (also referred to as “leucine zipper-like repeats”) comprising an 8 amino acid sequence initiating with, and ending with, an isoleucine or leucine. Most frequently, the HR2 region has just one leucine zipper like-motif, whereas the HR1 region has five leucine zipper-like motifs. Heptads and leucine zipper like-motifs contribute to formation of a coiled coil structure of gp41, and of a coiled coil structure of peptides derived from the HR regions. Generally, coiled coils are known to be comprised of two or more helices that wrap around each other in forming oligomers, with the hallmark of coiled coils being a heptad repeat of amino acids with a predominance of hydrophobic residues at the first (“a”) and fourth (“d”) positions, charged residues frequently at the fifth (“e”) and seventh (“g”) positions, and with the amino acids in the “a” position and “d” position being primary determinants that influence the oligomeric state and strand orientation (see, e.g., Akey et al., 2001, Biochemistry, 40:6352-60).

It was discovered that synthetic peptides derived from either the HR1 region (“HR1 peptides”) or HR2 region (“HR2 peptides”) of HIV gp41 inhibit transmission of HIV to host cells both in in vitro assays and in in vivo clinical studies (see, e.g., Wild et al., 1994, Proc. Natl. Acad. Sci. USA, 91:9770-9774; U.S. Pat. Nos. 5,464,933 and 5,656,480 licensed to the present assignee; and Kilby et al., 1998, Nature Med. 4:1302-1306). More particularly, HR1 peptides exemplified by DP107 (also known as T-21; SEQ ID NO:1) blocked infection of T cells with 50% effective concentration values (EC50) of 1 μg/ml (see, e.g., Lawless et al., 1996, Biochemistry, 35:13697-13708). Sedimentation equilibrium experiments indicated that, in solution, T-21 peptide exists in a monomer/dimer/tetramer equilibrium (e.g., at concentrations of 5 μM or less, with predominately tetramers at high concentrations of peptide (e.g., 10 μM or more). A structural interaction occurring between a HR2 peptide and HR1 peptide has been observed when HR1 peptide is tetrameric (Lawless et al., supra). However, the generally accepted model of gp41 suggests that the gp41 core exists as a six helix bundle comprised of three N-terminal (HR1) regions forming a parallel trimeric coiled coil, where three C-terminal (HR2) regions pack in an antiparallel orientation into the hydrophobic grooves on the surface of the trimeric coiled coil (see, e.g., Shu et al., 1999, Biochemistry 38:5378-5385; Root et al., 2001, Science 291:884-888, and U.S. Pat. No. 6,150,088).

Pioneering potent synthetic peptides which inhibit HIV membrane fusion, thereby preventing transmission of the virus to a host cell, have been described previously (see, e.g., U.S. Pat. Nos. 6,258,782 and 6,348,568 assigned to the present assignee). However, it is desirable to design and/or develop new synthetic peptides, peptide-mimetics or small molecules (chemicals) as next generation drugs to inhibit HIV membrane fusion. There are several techniques for identifying peptides or small molecules that inhibit HIV fusion. In one method, a compound may be identified as an HIV fusion inhibitor by its ability to inhibit the formation of DP107/DP178 complexes (see, e.g. U.S. Pat. No. ______, assigned to the present assignee). The basic principle of this method is that compounds which can interfere with the binding reaction between peptide DP107 (T-21) and peptide DP178 (T-20) often exhibit potent antiviral activity as fusion inhibitors. An assay using HR1 and HR2 peptides (N36 and C34 peptides) has also been described (see, e.g., U.S. Pat. No. 6,150,088). Care must be exercised as to the concentration of the reactants, as HR1 peptides have a tendency to aggregate in the absence of HR2 peptides (Eckert et al., 1999, Cell 99:103-115). Additionally, HR1 peptides predominate in solution in tetrameric form by themselves. Thus, in the absence of HR2 peptide, a non-trimeric structure is presented to compounds being evaluated for activity as fusion inhibitors. More desirable is a trimer comprised of three HR1 peptides, in evaluating compounds and producing drugs with activity as fusion inhibitors, since the x-ray crystallographic structure of the HR1/HR2 complex has indicated that, as part of gp41, the HR1 region forms a trimer to which three HR2 regions bind in forming a six-helix bundle in vivo.

In a method for identifying a compound that can bind to an HR1 peptide, and in an effort to force HR1 peptide into trimeric structures, N36 peptide sequence (a peptide of native sequence derived from the HR1 region of HIV gp41) was fused to a protein GCN4 which forms a trimeric coiled coil in creating a fusion protein (Eckert et al., 1999, Cell, 99:103-115). However, the GCN4 portion is trimeric, and contains an analogous groove to gp41; and hence, compounds can interact with the GCN4 portion of the fusion protein (rather than with N36 peptide) which can result in false positive leads for fusion inhibitors. Additionally, the GCN4 portion of the fusion protein is nearly 50% of the fusion protein; hence, representing a large portion of the total area to which a compound may interact. Lastly, since the GCN4 portion is not sequence derived from gp41, there is a question as to whether linking it to HR1 sequence such as N36 could alter binding by an HR2 peptide or compound which would normally bind to HR1 sequence. Further, a denaturant (guanidinium hydrochloride) at a concentration of less than 0.5M is necessary to keep such fusion protein in solution (Shu et al., 1999, Biochemistry 38:5378-5385) which could have adverse affects in an assay to screen small molecules that are potential fusion inhibitors. In another method for identifying a drug that inhibits HIV fusion, Chan et al. (U.S. Pat. No. 6,506,554) utilizes trimeric complexes of heterodimers, with each heterodimer comprised of an N36 peptide and a C34 peptide (column 9). A drug that can interact with a trimeric complex of heterodimers is suggested to be possibly useful to prevent six-helix bundle formation in the HIV fusion process. It is noted by the Chan et al., that N peptides by themselves tend to aggregate, thus the need to mix them with C34 peptides to get heterodimers prior to using them for screening.

Thus, there is a need for additional compounds which can interfere with the interaction of the various domains of gp41 involved in oligomerization and with the conformational changes of gp41 necessary to effect fusion, thereby inhibiting the fusion of HIV gp41 to a target cell membrane. Additionally, there is a need for a method, and base components for the method (particularly HR1 peptides self-assembled into trimeric form), used to identify and produce such compounds which inhibit the fusion of HIV to a target cell membrane. Preferably, this method would identify compounds which inhibit fusion by demonstrating their ability in vitro to bind to a trimer (formed of synthetic peptides having an amino acid sequence derived from HR1 region) from interacting with HR2 peptide.

### SUMMARY OF THE INVENTION

The present invention relates to the use of synthetic peptides derived from the HR1 region of gp41 wherein the peptides contain one or more site-specific amino acid substitutions (as compared to the native sequence of that HR1 region of gp41), in one or more of the plurality of heptads of the peptide, which unexpectedly result in a change in the oligomerization state in solution to self-association into a trimeric form; and the trimers formed therefrom. Also provided are trimers formed from synthetic peptide. These synthetic peptides, and trimers formed therefrom, are shown herein to retain the ability to bind peptides derived from the HR2 region. Hence, these synthetic peptides, and particularly the trimers formed therefrom, provide a novel means (e.g., in providing a trimer comprising a coiled coil) by which to identify compounds and/or produce molecules which inhibit the binding interaction between HR1 region and HR2 region involved in HIV gp41-mediated fusion events (e.g., the process of HIV fusing to a target cell membrane in the infection process).

Thus, in a primary object of the present invention, provided is a method for producing a drug that inhibits interaction between the HR1 region and HR2 region of HIV gp41 (preferably by one or more of: inhibiting complexing between HR1 and HR2, or disrupting an HR1-HR2 complex), using trimers as provided with the present invention. The method comprises contacting trimers, in the presence of a compound, with HR2 peptide (capable of complexing with HR1) under conditions and for a time sufficient to allow formation of a complex between the trimers and the HR2 peptide; and detecting the amount of complex formed; wherein inhibition or reduction of complex formation in the presence of the compound (e.g., as compared to the amount of complex formation detected in the absence of the compound) is indicative of ability of the compound to inhibit the interaction between the HR1 region and HR2 region of gp41; and further comprising contacting the compound with a pharmaceutically acceptable carrier in producing a drug.

In another object of the present invention, provided is a method for identifying a drug that inhibits interaction between the HR1 region and HR2 region of HIV gp41, the method comprising contacting trimers, formed from synthetic peptide provided with the present invention, in the presence of a drug and with HR2 peptide (capable of complexing with HR1) under conditions and for a time sufficient to allow formation of a complex between the trimers and the HR2 peptide; and detecting the amount of complex formed; wherein inhibition or reduction of complex formation in the presence of the drug (e.g., as compared to the amount of complex formation detected in the absence of the drug) is indicative of ability of the drug to inhibit the interaction between the HR1 region and HR2 region of gp41.

In a further object of the present invention, provided is a method for producing a drug that inhibits transmission of HIV to a target cell (e.g., a cell, selected from those cells known in the art to be infected by HIV), the method comprising contacting trimers, formed from synthetic peptide provided with the present invention, in the presence of a compound and with HR2 peptide (capable of complexing with HR1) under conditions and for a time sufficient to allow formation of a complex between the trimers and the HR2 peptide; and detecting the amount of complex formed; wherein inhibition or reduction of complex formation in the presence of the compound (e.g., as compared to the amount of complex formation detected in the absence of the compound) is indicative of ability of the compound to inhibit transmission of HIV to a target cell; and further comprising contacting the compound with a pharmaceutically acceptable carrier in producing a drug. In a preferred embodiment, inhibiting transmission of HIV to a target cell comprises inhibiting gp41-mediated fusion of HIV to a target cell.

In a further object of the present invention, provided is a method for identifying a compound that inhibits transmission of HIV to a target cell, the method comprising contacting trimers, formed from synthetic peptide provided with the present invention, in the presence of a compound and with HR2 peptide (capable of complexing with HR1) under conditions and for a time sufficient to allow formation of a complex between the trimers and the HR2 peptide; and detecting the amount of complex formed; wherein inhibition or reduction of complex formation in the presence of the compound (e.g., as compared to the amount of complex formation detected in the absence of the compound) is indicative of ability of the compound to inhibit gp41-mediated fusion of HIV to a target cell (or alternatively, that inhibits transmission of HIV to a target cell).

The above and other objects, features, and advantages of the present invention will be apparent in the following Detailed Description of the Invention when read in conjunction with accompanying drawings.

### BRIEF DESCRIPTION OF THE DRAWINGS

FIG. 1 is a schematic of HIV-1 gp41 showing the heptad repeat 1 region (HR1; SEQ ID NO:1) and heptad repeat 2 region (HR2) along with other functional regions of gp41. Exemplary amino acid sequences corresponding to HIV_{IIIB} HR1 and HR2 are shown for purposes of illustration. The amino acid residues are numbered according to their position in gp160, strain HIV_{IIIB}.

FIG. 2 shows a comparison of the sequences contained within the HR1 region of HIV-1 gp41 as determined from various laboratory strains and clinical isolates, wherein illustrated are some observed variations in amino acid sequence as indicated by the single letter amino acid code.

FIG. 3 shows a comparison between native sequence and illustrative synthetic peptides according to the present invention, wherein substitutions are underlined.

FIG. 4 is a graph showing a comparison of binding curves between HR2 peptide (having the amino acid sequence of SEQ ID NO:33) with native HR1 peptides having the amino acid sequences of SEQ ID NOs:24 or 23, or synthetic peptides having the amino acid sequences of SEQ ID NOs:32 or 36.

FIG. 5 is a graph showing use of a synthetic peptide provided with the present invention and an HR2 peptide as may be used in a method for producing a molecule that can inhibit binding between HR1 sequences and HR2 sequences.

### DETAILED DESCRIPTION OF THE INVENTION

### Definitions

The term “individual”, when used herein for purposes of the specification and claims, means a mammal, and preferably a human.

The term “target cell”, when used herein for purposes of the specification and claims, means a cell capable of being infected by HIV. Preferably, the cell is a human cell or are human cells; and more preferably, human cells capable of being infected by HIV via a process including membrane fusion.

The term “pharmaceutically acceptable carrier”, when used herein for purposes of the specification and claims, means a carrier medium that does not significantly alter the biological activity of the active ingredient (e.g., a synthetic peptide or trimer provided with the present invention, or a compound discovered according to a method of the present invention) to which it is added. As known to those skilled in the art, a suitable pharmaceutically acceptable carrier may comprise one or substances, including but not limited to, water, buffered water, saline, 0.3% glycine, aqueous alcohols, isotonic aqueous buffer; and may further include one or more substances such as water-soluble polymer, glycerol, polyethylene glycol, glycerin, oils, salts such as sodium, potassium, magnesium and ammonium, phosphonates, carbonate esters, lipids, fatty acids, saccharides, polysaccharides, glycoproteins (for enhanced stability), excipients, and preservatives and/or stabilizers (to increase shelf-life or as necessary and suitable for manufacture and distribution of the composition). A pharmaceutically acceptable carrier also comprises a diluent or buffer used in a method according to the present invention.

By the term “amino acid” is meant, for purposes of the specification and claims and in reference to the synthetic peptides provided with the present invention, to refer to a molecule that has at least one free amine group and at least one free carboxyl group. The amino acid may have more than one free amine group, or more than one free carboxyl group, or may further comprise one or more free chemical reactive groups other than an amine or a carboxyl group (e.g., a hydroxyl, a sulfhydryl, etc.). The amino acid may be a naturally occurring amino acid (e.g., L-amino acid), a non-naturally occurring amino acid (e.g., D-amino acid), a synthetic amino acid, a modified amino acid, an amino acid derivative, an amino acid precursor, and a conservative substitution. One skilled in the art would know that the choice of amino acids incorporated into a peptide will depend, in part, on the specific physical, chemical or biological characteristics required of the antiviral peptide. Such characteristics are determined, in part, by determination of trimeric structure (as described herein in more detail) and antiviral activity (as described herein in more detail). For example, the skilled artisan would know from the descriptions herein that amino acids in a synthetic peptide may be comprised of one or more of naturally occurring (L)-amino acid and non-naturally occurring (D)-amino acid. A preferred amino acid may be used to the exclusion of amino acids other than the preferred amino acid.

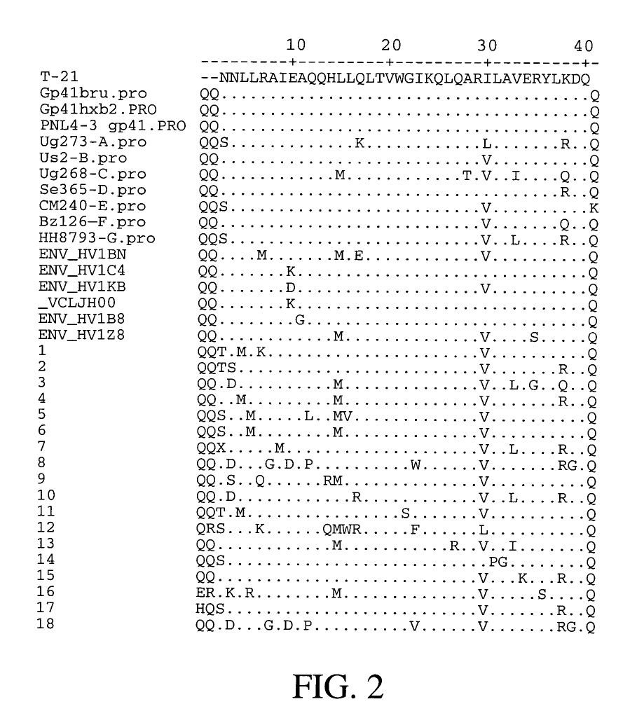

The term “amino acid substitution” is used in relation to the amino acid sequence of a native sequence of the HR1 region of HIV-1 gp41, and is also used in relation to amino acid sequence of a synthetic peptide provided with the present invention. The term “amino acid substitution” is used in relation to the native sequence, hereinafter for the purposes of the specification and claims, to mean one or more amino acids substitution in or to the amino sequence of the native sequence in producing a synthetic peptide that can self-assemble in solution into a trimeric form, and wherein the synthetic peptide can bind the HR2 region or a peptide derived thereform, as may be determined by the teachings herein and by using methods routine in the art. Likewise, when comparing synthetic peptides provided with the present invention, reference is often made to the amino acid sequence of one synthetic peptide as containing one or more additional amino acid substitutions when compared to the amino acid sequence of another synthetic peptide. Preferably, the amino acid substitution in the sequence of the synthetic peptide provided with the present invention ranges from 1 to 10 amino acids, and more preferably from 1 to 5 amino acids (the higher range being more desirable for longer peptides, e.g., about 40 or more amino acids in length). The amino acid substitution may comprise a “conservative substitution”. As known in the art “conservative substitution” is defined by aforementioned function, and includes substitutions of amino acids having substantially the same charge, size, hydrophilicity, and/or aromaticity as the amino acid replaced. Such substitutions are known to those of ordinary skill in the art to include, but are not limited to, glycine-alanine-valine; isoleucine-leucine; tryptophan-tyrosine; aspartic acid-glutamic acid; arginine-lysine; asparagine-glutamine; and serine-threonine. Such substitutions may also comprise polymorphisms, as illustrated in FIG. 2, at the various amino acid positions along the HR1 region found in laboratory and/or clinical isolates of HIV.

A “compound” is a term used hereinafter, for the purposes of the specification and claims, to mean a molecule which may include, but is not limited to, a small molecule chemical compound, a natural or synthetic peptide, a mimetic (e.g., peptidomimetic or other mimetic), a polypeptide, an aptamer, a polymer, an oligonucleotide, an antibody or fragment thereof, a polysaccharide, a carbohydrate-containing molecule, an enzyme, an agent, a macromolecule, a metabolite, a combination thereof, and the like. In a preferred embodiment, the compound is a therapeutically deliverable substance; and in a more preferred embodiment, the compound is a therapeutically deliverable substance that can inhibit binding between the HR1 and HR2 regions of HIV gp41, and in a further preferred embodiment, the compound is a therapeutically deliverable substance that can inhibit transmission of HIV to a target cell. A “drug” is a compound or composition of matter which, when administered to an individual (such as a human), induces a desired pharmacological (therapeutic, prophylatic, or a combination thereof) effect. In a preferred embodiment, a drug comprises a compound and a pharmaceutically acceptable carrier; and in a more preferred embodiment, the drug comprises a compound and a pharmaceutically acceptable carrier, wherein the drug can be administered in an amount effective to inhibit binding between the HR1 and HR2 regions of HIV gp41 (i.e., a desired pharmacological effect); and in a further preferred embodiment, the drug is a compound and a pharmaceutically acceptable carrier, wherein the drug can be administered in an amount effective to inhibit transmission of HIV to a target cell (i.e., a desired pharmacological effect), and more particularly, to inhibit HIV fusion to a target cell.

The term “native sequence”, when used herein for purposes of the specification and claims and in reference to the amino acid sequence of the HR1 region of HIV gp41, means a naturally occurring sequence found in laboratory HIV strains and/or HIV clinical isolates. Such sequences are readily available from public gene databases such as GenBank. For purposes of illustration, but not limitation, some of such native sequences are illustrated in FIG. 2, in which illustrative substitutions (e.g., polymorphisms) are noted in various amino acid positions in the amino acid sequence of that portion of the HR1 region of HIV-1 gp41 illustrated. More particularly, a “native sequence” means the amino acid sequence derived from the HR1 region of HIV gp41, which is identical to the amino acid sequence of the synthetic peptide to which the native sequence is compared, except that the native sequence lacks the one or more amino acid substitutions contained in the synthetic peptide which confer the ability of the synthetic peptide to self-assemble in solution into trimers. For example, SEQ ID NO:23 comprises the amino acid sequence of a native sequence when compared to SEQ ID NO:32 which comprises the amino acid sequence of a synthetic peptide provided with the present invention.

The term “reactive functionality”, when used herein for purposes of the specification and claims, means a chemical group or chemical moiety that is capable of forming a covalent bond and/or is protective (e.g., protects peptide derivatives from reacting with themselves). With respect to chemical groups, a reactive functionality is known to those skilled in the art to comprise a group that includes, but is not limited to, maleimide, thiol, carboxy, phosphoryl, acyl, hydroxyl, acetyl, hydrophobic, amido, dansyl, fluorenylmethyoxycarbonyl (Fmoc), t-butyloxycarbonyl (Boc), sulfo, a succinimide, a thiol-reactive, an amino-reactive, a carboxyl-reactive, and the like. A chemical moiety may comprise a linker. Linkers are known to refer to a compound or moeity that acts as a molecular bridge to operably link two different molecules (e.g., a wherein one portion of the linker binds to a peptide provided with the present invention, and wherein another portion of the linker binds to a macromolecular carrier or another antiviral peptide known to inhibit HIV transmission to a target cell). The two different molecules may be linked to the linker in a step-wise manner. There is no particular size or content limitations for the linker so long as it can fulfill its purpose as a molecular bridge. Linkers are known to those skilled in the art to include, but are not limited to, chemical chains, chemical compounds (e.g., reagents), and the like. The linkers may include, but are not limited to, homobifunctional linkers and heterobifunctional linkers. Heterobifunctional linkers, well known to those skilled in the art, contain one end having a first reactive functionality to specifically link a first molecule, and an opposite end having a second reactive functionality to specifically link to a second molecule. It will be evident to those skilled in the art that a variety of bifunctional or polyfunctional reagents, both homo- and hetero-functional (such as those described in the catalog of the Pierce Chemical Co., Rockford, Ill.), may be employed as a linker with respect to the present invention. Depending on such factors as the molecules to be linked, and the conditions in which the linking is performed, the linker may vary in length and composition for optimizing such properties as preservation of biological function stability, resistance to certain chemical and/or temperature parameters, and of sufficient stereo-selectivity or size. For example, the linker should not significantly interfere with the ability of the peptide (to which it is linked) to function as an inhibitor of either or both of HIV fusion and HIV transmission to a target cell. A preferred reactive functionality may be used with the present invention to the exclusion of a reactive functionality other than the preferred reactive functionality.

The term “macromolecular carrier”, when used herein for purposes of the specification and claims, means a molecule which is linked, joined, or fused (e.g., chemically or through recombinant means) to one or more synthetic peptides, or trimers formed therefrom, provided with the present invention, whereby the molecule is capable of conferring one or more of stability to the one or more peptides or trimers, or provides some other useful function as apparent to one skilled in the art (e.g., as an anchor for a solid phase assay format), in a method according to the present invention. Such macromolecular carriers are well known in the art to include, but are not limited to, polymers, carbohydrates, phospholipids, and lipid-fatty acid conjugates. Polymers typically used as macromolecular carriers include, but are not limited to, polylysines or poly(D-L-alanine)-poly(L-lysine)s, or polyols. A preferred polyol comprises a water-soluble poly(alkylene oxide) polymer, and can have a linear or branched chain. Suitable polyols include, but are not limited to, polyethylene glycol (PEG), polypropylene glycol (PPG), and PEG-PPG copolymers. A preferred polyol comprises PEG having an average molecular size selected from the range of from about 1,000 Daltons to about 20,000 Daltons. A preferred macromolecular carrier may be used with the present invention to the exclusion of a macromolecular carrier other than the preferred macromolecular carrier.

The term “synthetic”, in relation to a peptide provided with the present invention, is used hereinafter for the purposes of the specification and claims to mean that the peptide is produced by chemical synthesis, recombinant expression, biochemical or enzymatic fragmentation of a larger molecule, chemical cleavage of larger molecule, a combination of the foregoing or, in general, made by any other method in the art, and isolated. The term “isolated” when used in reference to a peptide, means that the synthetic peptide is substantially free of components which have not become part of the integral structure of the peptide itself; e.g., such as substantially free of cellular material or culture medium when produced by recombinant techniques, or substantially free of chemical precursors or other chemicals when chemically synthesized or produced using biochemical or chemical processes. With reference to a “synthetic peptide” which “self-assembles into trimers” (or a “trimeric form”) is meant that multiple molecules of synthetic peptide are present, and that such molecules of synthetic peptide self-assemble into trimers (e.g., a trimer being comprised of three molecules of synthetic peptide). With reference to a “synthetic peptide” which “self-assembles into trimers” (or a “trimeric form”) is meant that when multiple molecules of synthetic peptide are present in a solution (e.g., an aqueous solution, or physiological solution, or pharmaceutically acceptable carrier) such molecules of synthetic peptide can self-assemble into trimers, wherein a trimer is comprised of three molecules of synthetic peptide, and wherein trimers are the predominant (preferred) oligomeric form of the synthetic peptide (with a minority, if detectable at all, of oligomeric forms other than a trimer) present in the solution, as can be determined using standard methods known in the art.

As previously summarized herein, the hallmark of coiled coils is that they comprise a heptad repeat of amino acids with each heptad having a predominance of hydrophobic residues at the first (“a”) and fourth (“d”) positions, charged residues frequently at the fifth (“e”) and seventh (“g”) positions, and with the amino acids in the “a” position and “d” position being determinants that influence the oligomeric state and strand orientation (see, e.g., Akey et al., 2001, Biochemistry, 40:6352-60). The effect on stability and oligomerization state of a model coiled coil, by substituting various amino acids at the “a” position and at the “d” position, have been reported previously, wherein formation of a trimeric structure was particularly dependent on the substitution at the “d” position (see, e.g., Tripet et al., 2000, J. Mol. Biol. 300:377-402; and Wagschal et al., 1999, J. Mol. Biol. 285:785-803). With respect to peptides derived from the native sequence of the HR1 region of HIV gp41, such peptides self assemble predominantly in tetrameric form at higher concentrations of peptide, and destabilize into monomers at lower concentrations (nanomolar range or less) of peptide.

A synthetic peptide provided with the present invention comprises the following distinguishing and functional characteristics.

### A. Sequence

A synthetic peptide provided with the present invention is derived from the native sequence of the HR1 region of HIV-1 gp41 in that it comprises a sequence that includes a series of contiguous amino acids that are identical to a series of contiguous amino acids of the HR1 region (preferably, no less than 16 and no more than 60 amino acids of the HR1 region), except for one or more strategically placed amino acid substitutions which unexpectedly affect the oligomerization of the peptide such that the synthetic peptide self assembles into predominantly trimeric form when in solution. A discovery of the present invention is that there is a cluster of hydrophobic amino acids in the HIV-1 gp41 HR1 region, where one or more amino acid substitutions therein results in a change in oligomerization in forming a synthetic peptide provided with the present invention. This cluster of hydrophobic amino acids is characterized by two adjoining heptad repeats in which an amino acid substitution in the “g” position of the first heptad (closer to the N-terminal portion of the HR1 peptide as compared to the heptad it adjoins) and an amino acid substitution in the “c” position of the second heptad of the two adjoining heptads unexpectedly affects the oligomerization state of the synthetic peptide comprising such substitutions, as compared to an HR1 peptide of native sequence (e.g., without such substitutions). In another example, an amino acid substitution in such “c” position alone unexpectedly results in a change in the oligomerization state of a synthetic peptide comprising the substitution, as compared to an HR1 peptide of native sequence (e.g., without such substitution).

More particularly, and with reference to FIG. 2, in two adjoining heptads (abcdefgabcdefg), the cluster of hydrophobic amino acids comprises the sequence (single letter amino acid code) “IEAQQHLLQLTVWG” (amino acid residues in positions 24 to 37 of SEQ ID NO:1, or polymorphisms thereof, see, e.g., FIG. 2 for some illustrations of HR1 amino acid sequences that differ slightly from SEQ ID NO:1 as a result of viral polymorphisms). A motif of such cluster of hydrophobic amino acids, with the motif referred to hereinafter as the “hydrophobic domain” of the HR1 region, may be generally represented by the sequence “QHLLQLTVW” (amino acid residues in positions 28 to 36 of SEQ ID NO:1 or polymorphisms thereof) and comprising heptad repeat positions “efgabcdef”, or represented by the sequence “QHXXZLTVW” comprising heptad repeat positions “efgabcdef”, where X is typically leucine or methionine; and Z is typically glutamine, but may also be another amino acid such as lysine or glutamic acid. Thus, for example, a substitution in either the “c” position of the hydrophobic domain (e.g., at amino residue 33 of SEQ ID NO:1 or polymorphisms thereof) or in both the “g” position and the “c” position of this hydrophobic domain of the HR1 region (e.g., at amino acid residue 30 and amino residue 33, respectively, of SEQ ID NO:1 or polymorphisms thereof) alters the oligimerization state of the resultant synthetic peptide when in solution. It will be apparent to one skilled in the art that any peptide derived from the native sequence of the HR1 region of HIV gp41 which has antiviral activity (as can be determined using methods standard in the art without undue experimentation), and which contains all or a portion (e.g., no less than “QHLL” or “QHXX” at the carboxy terminus of the native sequence) of the hydrophobic domain, can be used as a native sequence into which one or more amino acid substitutions in the hydrophobic domain may be introduced to produce a synthetic peptide provided with the present invention. For purposes of illustration, such HR1 peptides derived from the native sequence, and from which a synthetic peptide may be produced, may include, but are not limited to, SEQ ID NOs:1 to 28.

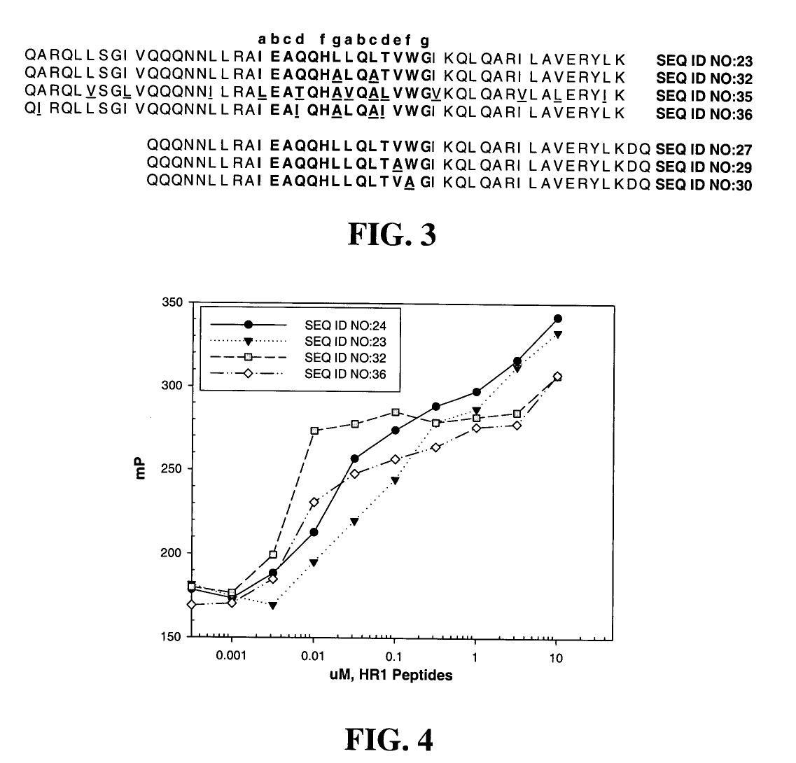

For example, a synthetic peptide provided with the present invention comprises a heptad repeat that differs from the native sequence of a heptad repeat of the HR1 region of HIV-1 gp41 in that in the hydrophobic domain of the synthetic peptide provided with the present invention, there is an amino acid substitution in either the “c” position, or in both the “g” position and the “c” position. More particularly, it is noted with significance that native sequences of the HR1 region of gp41 appear strikingly conserved in having a bulky, buried, nonpolar amino acid consisting of either a leucine or methionine in the “g” position of the hydrophobic domain and a leucine in the “c” position of the hydrophobic domain (see, e.g., FIG. 2). The synthetic peptides used with the present invention, as compared to the native sequence, comprises a substitution in either the “c” position of the hydrophobic domain or in both the “g” position and “c” position of the hydrophobic domain, with an amino acid, preferably with a hydrophobic amino acid other than methionine and leucine, more preferably with a hydrophobic amino acid with uncharged, nonpolar head groups (e.g., alanine, valine, and the like), and more preferably with an amino acid comprising alanine. Thus, the synthetic peptide comprises a heptad repeat containing this hydrophobic domain and one or more amino acid substitutions therein (at the “c” position or at both the “g” and “c” positions), which one or more amino acid substitutions confers on the synthetic peptide the ability to self-assemble in solution to a predominately trimeric form as can be determined using methods standard in the art (see, e.g., Example 1 and Table 1). The number of heptads in the heptad repeat may vary, depending on the length of the synthetic peptide used with the present invention. However, preferably, the synthetic peptide will comprise a heptad repeat comprised of at least 3 complete, contiguous heptads, and more preferably will comprise a heptad repeat of at least 5 complete, contiguous heptads (e.g., see FIG. 3). A synthetic peptide used with the present invention, in addition to the one or more substitutions at the “g” position and/or “c” position of the hydrophobic domain, may also comprise an additional amino acid substitution in one or more of the heptads of the peptide in a position selected from the group consisting of an “a” positions, a “d” position, a “b” position, or a combination thereof (see, e.g., FIG. 3, and Example 2), as compared to the native sequence of HR1 of HIV-1 gp41. As illustrated herein, such additional amino acid substitutions may range in number from about 1 to about 15 amino acid substitutions, with the synthetic peptide still retaining the ability to self-associate in solution into trimers, and demonstrate antiviral activity against HIV-1, as compared to native sequence.

In another embodiment, a synthetic peptide used with the present invention may comprise an amino acid substitution in either or both of the C-terminal “e” position and “f” position of the hydrophobic domain of the HR1 region (e.g., amino acid residue 35 and/or amino 36 of SEQ ID NO:1 or a polymorphism thereof); comprising a substitution of either or both of the valine and tryptophan (see, e.g., FIG. 3, SEQ ID NO:29-30) as compared to the native sequence of the HR1 region of HIV-1 gp41. It is noted with significance, that amino acids at these two positions (e.g., the valine and tryptophan) appear to be highly conserved amongst different strains of HIV-1 (see, e.g., FIG. 2). As compared to the native sequence, a synthetic peptide used with the present invention comprises a substitution in either or both the C terminal “e” position and “f” position of the hydrophobic domain with an amino acid, preferably with a hydrophobic amino acid other than valine (with respect to the “e” position) and tryptophan (with respect to the “f” position), more preferably with a hydrophobic amino acid with uncharged, nonpolar head groups (e.g., alanine and the like), and more preferably with alanine, which amino acid substitution(s) confers on the synthetic peptide the ability to self-assemble in solution to a predominately trimeric form as can be determined using methods standard in the art. In a preferred embodiment, and as compared to the native sequence, a synthetic peptide used with the present invention comprises a combination of amino acid substitutions as described in this section A (e.g., a substitution in the “c” position or in both the “g” and “c” position, and in one or more of the C-terminal “e” position and “f” position, in the hydrophobic domain; a substitution in the “c” position or in both the “g” and “c” position, and in one or more of the C-terminal “e” position and “f” position, of the hydrophobic domain, and further comprising substitutions in one or more of the heptads of the peptide in one or more of an “a” position, “b” position, and “d” position; a substitution in either or both the C-terminal “e” position and “f” position of the hydrophobic domain, and further comprising substitutions in one or more of the heptads of the peptides in one or more of an “a” position, a “b” position, and a “d” positions; etc.).

### B. Oligomerization

Peptides derived from the HR1 region of HIV gp41, by themselves, have been reported to exist in solution in a monomer/ dimer/tetramer equilibrium, and can self-assemble into predominately tetramers, depending in the concentration of the peptide. Since the synthetic peptides provided with the present invention can self-assemble into trimers (the preferred oligomeric form upon self-assembly), a primary advantage of the methods of the present invention (as compared to using HR1 peptides previously described in the art) is that a native-like trimer, formed by a synthetic peptide provided with the present invention, is presented for binding interactions with HR2 of HIV gp41 in a process of identifying and/or producing compounds or drugs which inhibit the binding interaction between the HR1 and HR2 regions of the HIV gp41. Since peptide models focus on the “a” position and “d” position in the heptad repeat as being the determinants that determine oligomerization, it is unexpected that a peptide can preferably self-assemble as trimers as a result of substitutions in either the “c” position or in both the “g” position and the “c” position of the hydrophobic domain, and/or in one or more of the C-terminal “e” position and “f” position of the hydrophobic domain, of the native sequence of the HR1 region of HIV-1 gp41. The discovery made by the present inventors is that there are specific, key residues in the amino acid sequence of the HR1 region of HIV gp41 (other than at the “a” and “d” positions) that may govern oligomerization; and further, that substitution of one or several of these key residues can result in a synthetic peptide that self-assembles into trimers as a preferred oligomeric form (e.g., as opposed to a tetramer). A trimer, employed in a method according to the present invention, is formed in solution (e.g., in a physiological solution or other aqueous environment) by self-assembly of synthetic peptide provided with the present invention.

### C. Size

A synthetic peptide employed in the present invention may comprise a sequence of no less than about 18 amino acids and no more than about 60 amino acid residues in length, and preferably no less than 30 amino acids and no more than about 51 amino acids in length, and more preferably no less than about 41 amino acids and no more than about 51 amino acids in length. Preferably, the synthetic peptide comprises a contiguous sequence consisting of at least 20 amino acid residues within the amino acid residues 23 to 59 of SEQ ID NO:1, and more preferably comprises amino acids “IEAQQHLLQLTVWG” (e.g., amino acids 24 to 37 of SEQ ID NO:1; or polymorphisms thereof), and further comprising one or more amino acid substitutions in the hydrophobic domain thereof, as this portion of the HR1 region and such substitutions in the hydrophobic domain have been found pursuant to this invention to influence the oligomeric form described herein in more detail. As also described herein in more detail, a synthetic peptide may further comprise a macromolecular carrier.

### D. Ability to Bind to HR2 Peptides

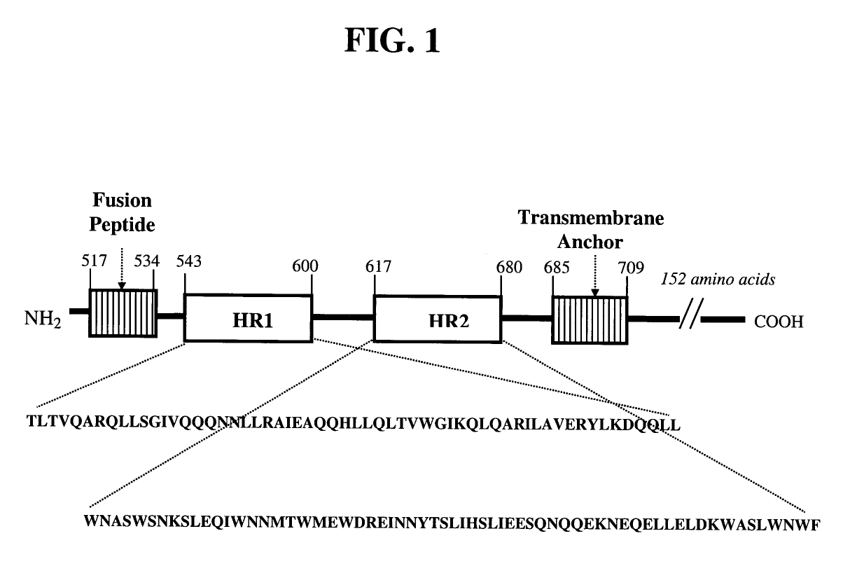

It is an important feature for the method of the present invention that a synthetic peptide (preferably in self-assembled trimeric form) employed in the present invention to be capable of complexing with a peptide derived from the HR2 region. Peptides derived from the HR2 region (“HR2 peptides”) which are known to complex with HR1 amino acid sequences are well known in the art (see, e.g., U.S. Pat. No. 5,464,933; see also FIG. 1). More specifically, a synthetic peptide provided with the present invention self-assembles into predominantly trimers, and trimers formed are capable of complexing with such HR2 peptide. Such complexing can be detected using methods standard in the art, and as described herein in more detail.

As described herein in more detail, a synthetic peptide provided with the present invention may further comprise: (a) one or more reactive functionalities (e.g., at either the C-terminal end, or N-terminal end, or a combination thereof (both the C-terminal end and N-terminal end)); (b) a pharmaceutically acceptable carrier; (c) a macromolecular carrier; (d) or a combination thereof. Likewise, a trimer according provided with the present invention may further comprise (a) one or more reactive functionalities; (b) a pharmaceutically acceptable carrier; (c) a macromolecular carrier; (d) or a combination thereof.

The present invention is illustrated in the following examples, which are not intended to be limiting.

### EXAMPLE 1

Peptides were synthesized on a peptide synthesizer using standard solid-phase synthesis techniques and using standard FMOC peptide chemistry. In this example, the synthetic peptides further comprised reactive functionalities; i.e., were blocked at the N-terminus by an acetyl group and at the C-terminus by an amide group. After cleavage from the resin, the peptides were precipitated, and the precipitate was lyophilized. The peptides were then purified using reverse-phase high performance liquid chromatography; and peptide identity was confirmed with electrospray mass spectrometry. In one embodiment, synthetic peptide, as exemplified by the synthetic peptide having the amino acid sequence of SEQ ID NO:32, was synthesized to comprise amino acid substitutions at the “g” position and “c” position in the hydrophobic domain. The synthetic peptide having the amino acid sequence of SEQ ID NO:32 was derived from the HR1 region of HIV-1 gp41 in that it comprises a sequence corresponding to amino acid residues 5 to 53 of SEQ ID NO:1 except, however, amino acid substitutions were made in amino acid residues corresponding to the “g” position and the “c” position of the hydrophobic domain (each a leucine in the HIV_{IIIB} sequence). Thus, the synthetic peptide having the amino acid sequence of SEQ ID NO:32 is 49 amino acids in length, and comprises a heptad repeat of 6 complete heptads with 5 leucine zipper-like motifs.

A similar embodiment of the present invention is a synthetic peptide having the amino acid sequence of SEQ ID NO:34, which has the same sequence as the synthetic peptide having the amino acid sequence of SEQ ID NO:32 except, however, that two amino acids (an aspartic acid and glutamine) were added to the C-terminus. In yet another illustration of the present invention, synthetic peptide, as exemplified by the synthetic peptide having the amino acid sequence of SEQ ID NO:47, was synthesized to comprise amino acid substitution at only the “c” position in the hydrophobic domain. The synthetic peptide having the amino acid sequence of SEQ ID NO:48 was derived from the HR1 region of HIV-1 gp41 in that it comprises a sequence corresponding to amino acid residues 5 to 55 of SEQ ID NO:1 except, however, amino acid substitution was made in amino acid residue corresponding to the “c” position of the hydrophobic domain (amino acid residue in position 33 of SEQ ID NO:1; e.g., a leucine in the HIV_{IIIB} sequence). Thus, the synthetic peptide having the amino acid sequence of SEQ ID NO:47 is 51 amino acids in length, and comprises a heptad repeat of 6 complete heptads with 5 leucine zipper-like motifs. A similar embodiment of the present invention is a synthetic peptide having the amino acid sequence of SEQ ID NO:48, and comprising a sequence corresponding to amino acid residues 15 to 55 of SEQ ID NO:1 except, however, amino acid substitution was made in amino acid residue corresponding to the “c” position of the hydrophobic domain (amino acid residue in position 33 of SEQ ID NO:1; e.g., a leucine in the HIV_{IIIB} sequence). These synthetic peptides were compared to an HR1 peptide, derived from native sequence of the HR1 region and which has the amino acid sequence of SEQ ID NO:23, for oligomerization state, helicity, stability, and binding to HR2 peptide (such as an HR2 peptide having the amino acid sequence of SEQ ID NO:33) using the following methods.

Oligomerization state was assessed by using methods standard in the art for performing sedimentation equilibrium experiments and for analysis of the resultant data. In that regard, sedimentation equilibrium experiments were performed over a concentration range of synthetic peptide (1 μM to 100 μM) as follows. Briefly, six-channel cells (12 mm optical path length) were used with an An-60 Ti rotor operated at 13,500 rpm, 18,000 rpm, 22,000 rpm, and 35,000 rpm) in an analytical ultracentrifuge at 4C. The cell radii were scanned using 0.001 cm steps with 10 averages/scan at 3 different wavelengths (235 nm, 240 nm, 280 nm). Data obtained with solutions containing synthetic peptide was analyzed using a single species model to determine a weight-averaged molecular weight (MW). Diagnostic plots of M_{W}/ M_{W0} vs. rpm/rpm₀ and M_{W} vs. radial concentration were used to test for sample homogeneity. When systemic residuals or a M_{W} higher than monomer molecular weight indicated the presence of self-association, associative models were investigated. The suitability of a model (goodness of fit) was judged by the trends observed in the residuals.

Helicity was assessed by circular dichroism (“CD”) as follows. Briefly, CD spectra were obtained using a spectrometer equipped with a thermoelectric temperature controller. The spectra was obtained at 25° C. with 0.5 nanometer (nm) steps from 200 to 260 nm, with a 1.5 nm bandwith, and a typical averaging time of 4 seconds/step. After the cell/buffer blank was subtracted, spectra were smoothed using a third-order least-squares polynomial fit with a conservative window size to give random residuals. Raw ellipticity values were converted to mean residue ellipticity using standard methods, and plotted was the wavelength (from 200 to 260 nm) versus [θ]×10⁻³ (degrees cm²/dmol). Percent helicity values were then calculated using standard methods. Assessment of thermal stability was performed by monitoring the change in CD signal at 222 nm as temperature was raised in 2° C. steps, with 1 minute equilibration times. The thermal stability for each sample (e.g., synthetic peptide) was represented by the transition temperature (Tm) determined by the derivative method.

In determining antiviral activity (e.g., as one measure of a synthetic peptide binding gp41 in a region that inhibits transmission of HIV to a target cell, thus making the synthetic peptide useful in a method according to the present invention for identifying compounds or producing drugs having antiviral activity against HIV), used was an in vitro assay which has been shown, by data generated using peptides derived from the HR regions of HIV gp41, to be predictive of antiviral activity observed in vivo. More particularly, antiviral activity observed using an in vitro infectivity assay (“Magi-CCR5 infectivity assay”; see, e.g., U.S. Pat. No. 6,258,782) has been shown to reasonably correlate to antiviral activity observed in vivo for the same HIV gp41 derived peptides (see, e.g., Kilby et al., 1998, Nature Med. 4:1302-1307). These assays score for reduction of infectious virus titer employing the indicator cell lines MAGI or the CCR5 expressing derivative cMAGI. Both cell lines exploit the ability of HIV-1 tat to transactivate the expression of a β-galactosidase reporter gene driven by the HIV-LTR. The β-gal reporter has been modified to localize in the nucleus and can be detected with the X-gal substrate as intense nuclear staining within a few days of infection. The number of stained nuclei can thus be interpreted as equal to the number of infectious virions in the challenge inoculum if there is only one round of infection prior to staining. Infected cells are enumerated using a CCD-imager and both primary and laboratory adapted isolates show a linear relationship between virus input and the number of infected cells visualized by the imager. In the MAGI and cMAGI assays, a 50% reduction in infectious titer (Vn/Vo=0.5) is significant, and provides the primary cutoff value for assessing antiviral activity (“IC50” is defined as the dilution resulting in a 50% reduction in infectious virus titer). A secondary cutoff of Vn/Vo=0.1, corresponding to a 90% reduction in infectious titer is also assessed (“IC90”). Peptides tested for antiviral activity were diluted into various concentrations, and tested in duplicate or triplicate against an HIV inoculum adjusted to yield approximately 1500-2000 infected cells/well of a 48 well microtiter plate. The peptide (in the respective dilution) was added to the cMAGI or MAGI cells, followed by the virus inocula; and 24 hours later, an inhibitor of infection and cell-cell fusion (e.g., T-20) was added to prevent secondary rounds of HIV infection and cell-cell virus spread. The cells were cultured for 2 more days, and then fixed and stained with the X-gal substrate to detect HIV-infected cells. The number of infected cells for each control and peptide dilution was determined with the CCD-imager, and then the IC50 and IC90 were calculated (expressed in μg/ml).

Several assays were used to assess the ability of the synthetic peptides/trimers employed with the present invention to retain the ability to complex with peptides derived from the HR2 region of HIV-1 gp41. These include a protein chip assay (using Biacore analysis) in which surface plasmon resonance is used to detect the binding, and kinetics thereof, between a synthetic peptide and an HR2 peptide; and a fluorescence polarization assay. In the fluorescence polarization assay, a detectable moiety comprising a fluorescent dye (e.g., rhodamine and/or fluorescein) was used to label HR2 peptide, and the assay was then allowed to proceed under sufficient conditions and for a sufficient period of time for labeled peptide to bind to HR1 peptide, or synthetic peptide (or trimer formed therefrom). Fluorescence intensity of polarized light was then measured with a detection system comprising a spectrophotometer, and the amount of polarized light was calculated (units=mP). As illustrated in FIG. 4, and exemplified by synthetic peptide having the amino acid sequence of SEQ ID NO:32, the synthetic peptides supplied in the present invention (trimers formed therefrom) retain the ability to complex with peptides derived from the HR2 region of HIV-1 gp41. In some instances throughout the Examples illustrated herein, such ability to complex with HR2 peptides as demonstrated by this binding assay is denoted as “yes” with reference to such binding activity.

Comparison of synthetic peptides (illustrated as SEQ ID NO:32 and SEQ ID NO:34) with HR1 peptides of native sequence (illustrated as SEQ ID NO:23 and SEQ ID NO:24,respectively) is illustrated in Table 1. As shown in Table 1, as compared to a respective peptide having the native sequence of HR1 (e.g., SEQ ID NOs: 23 & 24), a synthetic peptide provided with the present invention (e.g., as exemplified by SEQ ID NOs:32, 34, 47, and 48) resulted in an alteration in the oligomerization state. Additionally, as shown in Table 1 (and subsequent Tables herein), a synthetic peptide (trimers formed therefrom) provided with the present invention can demonstrate relatively good binding to HR2 peptide, and a significant increase in antiviral activity (e.g., 3 fold or greater increase in potency, as observed by a ⅓ or greater decrease in either or both of IC50 and IC90) as compared to an HR1 peptide of native sequence.

TABLE 1
Biophysical and antiviral Parameters

| Parameter | SEQ ID NO:23 | SEQ ID NO:24 | SEQ ID NO:32 | SEQ ID NO:34 | SEQ ID NO:47 | SEQ ID NO:48 |
|---|---|---|---|---|---|---|
| Oligomerization | Aggregates | Best modeled | Best modeled | Best modeled | Best modeled as | Best modeled |
| | | As a tetramer | as a trimeric | as a trimeric | a trimeric | as a trimeric |
| | | | species* | species* | species* | species* |
| Helicity (at 10 μM, | 83% | 74% | 87% | 100% | 68% | 72% |
| 25° C.) | | | | | | |
| Stability(at 10 μM) | Tₘ 83° C. | Tₘ 82° C. | Tₘ 69° C. | Tₘ 71° C. | Tₘ 77° C. | Tₘ 68° C. |
| HR2 peptide | Yes | yes; 1.2 × 10⁶/ | Yes; 6.1 × 10⁵/ | Yes | Yes | Yes |
| binding | | 1.2 × 10³/ | 1.9 × 10³/ | | | |
| K on/K off/Kd | | 1.1 nM | 3.1 nM | | | |
| Antiviral activity | | | | | | |
| Against HIV IIIB | | | | | | |
| (μg/ml) | | | | | | |
| IC50 | 4.69 | 3.73 | 0.59 | 0.19 | 0.61 | 0.89 |
| IC90 | 23.24 | 12.301 | 1.90 | 0.62 | | |

*with only a small fraction (e.g., between about 1 to about 10%) of higher order oligomer (e.g., hexamer) and therefore is considered to self assemble into trimers.

It was an unexpected result that by replacing the leucine at the aforementioned “c” position of the hydrophobic domain (e.g., as exemplified by synthetic peptides having the amino acid sequence of SEQ ID NOs:47 & 48), or replacing the leucine or methionine at the aforementioned “g” position of the hydrophobic domain and the leucine at the aforementioned “c” position of the hydrophobic domain (e.g., as exemplified by synthetic peptides having the amino acid sequence of SEQ ID NOs:32 & 34) with a less bulky amino acid (an amino acid having a less bulky side chain or R group than leucine or methionine), altered in the synthetic peptide derived from the HR1 region can be (a) the oligomerization state of such synthetic peptide, in that it can self-assemble in solution to predominately trimers rather than tetramers (e.g., the latter being the predominant oligomeric state in solution of respective native sequence having the amino acid sequences of SEQ ID NOs:23 & 24); and (b) antiviral activity to a significant improvement (increase) in antiviral activity against HIV isolates (e.g., compare the statistically significant difference in the antiviral activity of a trimer of a synthetic peptide having the amino acid sequence of any one of SEQ ID NOs:32, 34, 47 or 48 versus a tetramer of an HR1 peptide of native sequence shown in SEQ ID NO:24). Amino acid residues which are less bulky than leucine and methionine are known to those skilled in the art to include, but are not limited to, alanine, glycine, valine, serine, threonine, and the like. Also, one skilled in the art would appreciate from the data presented in Table 1, that the synthetic peptides provided with the present invention have utility in a method for identifying or producing a compound or drug. More particularly, the synthetic peptide can self-assemble into trimers, thereby presenting a structure like that of the native trimeric core of the HR1 region of HIV gp41 to which can bind molecules (compounds and/or drugs) having binding specificity for such structure. Since the trimers employed in the method according to the present invention retain the ability to complex with HR2 peptide, provided is the means to evaluate the ability of a molecule to inhibit the binding interaction between the HR1 and HR2 regions of HIV gp41 (e.g., by contacting trimer provided with the present invention, in the presence of HR2 peptide, with the molecule). More particularly, the synthetic peptides self assemble into trimers, and the trimers, when used in a method according to the present invention, provide a structure in vitro which mimics the trimers formed by the HR1 region of gp41 in vivo. Thus, a method of the present invention utilizing such trimers in vitro provide a means by which compounds can be identified or drugs produced which can inhibit/prevent six helix bundle formation between the trimers formed by the interaction of HR1 region of gp41 and trimer formed form the HR2 region of gp41 in forming a six-helix bundle in vivo.

Further, as illustrated in FIG. 4, in a fluorescence polarization assay to detect binding, the synthetic peptides (or trimers formed therefrom) provided with the present invention may exhibit a cooperative transition at a narrow concentration range, beyond which no significant change is observed. As exemplified using trimers formed from synthetic peptide having the sequence of SEQ ID NO:32, the sharpness of the transition enables more sensitive and accurate detection of a molecule (compound and/or drug) that can inhibit the binding between HR1 and HR2 regions of HIV gp41, as compared to a method using a peptide derived from the native sequence of HR1 (see FIG. 4, and the relative absence of plateaus defining a transition for SEQ ID NOs: 24 & 23, respectively). Accordingly, the method according to the present invention represents significant advantages in identifying or producing a molecule that can inhibit the binding between HR1 and HR2 regions of HIV gp41. Additionally, for the reasons provided above, the method according to the present invention may provide a means for discovery of a different profile (e.g., molecular structure, binding ability, and the like) of molecules when compared to molecules identified in a method using native sequence HR1 peptide and native sequence HR2 peptide as binding partners.

### EXAMPLE 2

In another embodiment, synthetic peptides, exemplified by the peptides having the amino acid sequences of SEQ ID NOs:35-40, were synthesized using the methods outlined in Example 1 herein to comprise amino acid substitutions at the “g” position and “c” position in the hydrophobic domain of the HR1 region of HIV-1 gp41, and additionally comprised an amino acid substitution in either (a) the “a” position of at least one of the heptads of the plurality of heptads comprising the peptide; or (b) an amino acid substitution in the “d” position of at least one of the heptads of the plurality of heptads comprising the peptide; or (c) a combination thereof (i.e., a combination of (a) and (b)). Where there is the combination thereof, the substitution in the “a” position and the substitution in the “d” position may be in the same heptad of the peptide, and/or may be in different heptads of the peptide. In a preferred embodiment, the substitution in either the “a” position and/or the “d” position is with an amino acid that are known to the art to stabilize the structure of a synthetic peptide; i.e., that induce the formation of a specific secondary structure (a coiled coil oligomer, for example; e.g., substitution of an amino acid other than Leu or lie with Leu or lie in forming additional leucine zipper-like motifs as compared to native sequence before such amino acid substitution).

A. This example illustrates a synthetic peptide provided with the present invention which, in addition to having amino acid substitutions in the “g” and “c” positions of the hydrophobic domain of the HR1 region of HIV-1 gp41, comprises an amino acid substitution in the “a” position of at least one of the heptads of the plurality of heptads comprising the peptide, and comprises an amino acid substitution in the “d” position of at least one of the heptads of the plurality of heptads comprising the peptide. For example, a synthetic peptide having the amino acid sequence of SEQ ID NO:35 comprises the same amino acid sequence as a synthetic peptide having the amino acid sequence of SEQ ID NO:32, except, however, amino acid substitutions were made in amino acids corresponding to residue positions 6, 20, 27, 34, 41, and 48 (e.g., in the “a” position of at least one heptad of the plurality of heptads), and to residue positions 9, 16, 23, 30, and 44 (e.g., in the “d” position of at least one heptad of the plurality of heptads), of SEQ ID NO:32. Thus, the synthetic peptide having the amino acid sequence of SEQ ID NO:35 comprises 49 amino acids in length and comprises a heptad repeat of 6 complete heptads but with only 3 leucine zipper-like motifs (as compared to 5 leucine zipper-like motifs for SEQ ID NO:32). Unexpectedly, the peptide having the amino acid sequence of SEQ ID NO:35 demonstrates a higher degree of stability as compared to that of the synthetic peptide having the amino acid sequence of SEQ ID NO:32 (see, Table 2).

In another example, the synthetic peptide having the amino acid sequence of SEQ ID NO:37 comprises the same amino acid sequence as the synthetic peptide with the amino acid sequence of SEQ ID NO:35 (an amino acid substitution in one or more “a” positions and in one or more “d”positions) except for one additional substitution at amino acid residue position 35 of SEQ ID NO:35 (e.g., in a “b” position of a heptad, whereby the arginine is substituted for a lysine). Thus, the synthetic peptide having the amino acid sequence of SEQ ID NO:37 comprises 49 amino acids in length and comprises a heptad repeat of 6 complete heptads and 3 leucine zipper-like motifs. Although synthetic peptides having the respective amino acid sequence of SEQ ID NO:35 and 37 vary by only one residue, as shown in Table 2, the helicity and stability are increased when the arginine residue is substituted for the lysine residue.

In another example, the synthetic peptide having the amino acid sequence of SEQ ID NO:38 comprises the same amino acid sequence as SEQ ID NO:32, except, however, substitutions were made in amino acid residue positions corresponding to residues 27, 34, 41, and 48 (e.g., in the “a” position of at least one heptad in the plurality of heptads) as well as amino acid residue positions corresponding to residues 23, 30, and 44 (e.g. in the “d” position of at least one heptad in the plurality of heptads), of SEQ ID NO:32. In addition, two amino acid residues, an aspartic acid and a glutamine, were added to the C-terminus, so that the synthetic peptide having the amino acid sequence of SEQ ID NO:38 comprises 51 amino acid residues. This synthetic peptide comprises a heptad repeat of 6 complete heptads and 3 leucine zipper-like motifs.

In yet another example, the synthetic peptide having the amino acid sequence of SEQ ID NO:39 has the same amino acid sequence as the synthetic peptide having the amino acid sequence of SEQ ID NO:32, except, however, substitutions were made at the amino acid residue positions corresponding to residues 6, 20, 41, and 48 (e.g., in the “a” position of at least one heptad in a plurality of heptads), and in amino acid residue positions corresponding to residues 9, 16, and 44 (e.g., in the “d” position of at least one heptad in a plurality of heptads) of SEQ ID NO:32; and additionally, an aspartic acid and glutamine were added to the C-terminus. Thus, the synthetic peptide having the amino acid sequence of SEQ ID NO:39 comprises a total length of 51 amino acid residues with 6 complete heptads and 4 leucine zipper-like motifs.

Such illustrative synthetic peptides (having the respective amino acid sequences of SEQ ID NOs:35, and 37-39) were compared to a peptide derived from that native sequence of the HR1 region without any amino acid substitutions (see, FIG. 3, SEQ ID NO:23) for oligomerization state, helicity, stability, antiviral activity, and binding to HR2 peptide (e.g., SEQ ID NO:33) using methods as previously described in more detail in Example 1 herein. The comparisons of these illustrative synthetic peptides provided with the present invention to an HR1 peptide of native sequence are illustrated in Table 2.

TABLE 2
Biophysical and antiviral Parameters

| | SEQ ID | SEQ ID | SEQ ID | SEQ ID | SEQ ID |
|---|---|---|---|---|---|
| Parameter | NO:23 | NO:35 | NO:37 | NO:38 | NO:39 |
| Oligomerization | Aggregates | Trimer | Trimer* | Trimer* | Trimer* |
| Helicity | 83% | 78% | 85% | 79% | 63% |
| (10 μM, 25° C.) | | | | | |
| Stability | Tₘ 83° C. | Tₘ >97° C. | Tₘ >97° C. | Tₘ 90° C. | Tₘ 68° C. |
| (at 10 μM) | | | | | |
| HR2 peptide | Yes | Yes | Yes | Yes | Yes |
| Binding | | 1.5 × 10⁶/ | | | |
| K on/K off/Kd | | 0.1 × 10⁻³/ | | | |
| | | 21.8 nM | | | |
| Antiviral activity | | | | | |
| against HIV IIIB | | | | | |
| (μg/ml) | | | | | |
| IC50 | 4.69 | 0.14 | <0.78 | 0.18 | 0.26 |
| IC90 | 23.24 | 0.69 | | 0.88 | 1.20 |

*Best modeled as a trimer with only a small fraction (e.g., about 1 to about 10%) of higher order oligomer (e.g., hexamer), and therefore, is considered to self assemble predominately into trimers.

B. This example illustrates a synthetic peptide provided with the present invention which, in addition to having amino acid substitutions in the “g” and “c” positions of the hydrophobic domain of the HR1 region of HIV-1 gp41, comprises an amino acid substitution in the “d” position of at least one of the heptads of the plurality of heptads comprising the synthetic peptide. Illustrating this example, a synthetic peptide having the amino acid sequence of SEQ ID NO:36 comprises the same amino acid sequence as SEQ ID NO:32, except, however, amino acid substitutions were made in amino acid residue positions corresponding to residues 2, 23, and 30 (e.g., in the “d” position of at least one heptad of the plurality of heptads) of SEQ ID NO:32. Thus, the synthetic peptide having the amino acid sequence of SEQ ID NO:36 comprises 49 amino acids in length and comprises a heptad repeat of 6 complete heptads but with at least 6 leucine zipper-like motifs (as compared to 5 leucine zipper-like motifs for the synthetic peptide having the amino acid sequence of SEQ ID NO:32 and 3 leucine zipper-like motifs for the synthetic peptide having the amino acid sequence of SEQ ID NO:35). Such illustrative synthetic peptide (having the amino acid sequence of SEQ ID NO:36) was compared to a peptide derived from that native sequence of the HR1 region without any amino acid substitutions (SEQ ID NO:23) for oligomerization state, helicity, stability, antiviral activity, and binding to HR2 peptide, using methods as previously described in more detail in Example 1 herein. The comparisons of this illustrative synthetic peptide provided with the present invention to a peptide of native sequence is illustrated in Table 3.

TABLE 3
Biophysical and antiviral Parameters

| | SEQ ID | SEQ ID |
|---|---|---|
| Parameter | NO:23 | NO:36 |
| Oligomerization | Aggregates | Trimer* |
| Helicity (10 μM, 25° C.) | 83% | 90% |
| Stability (at 10 μM) | Tₘ | Tₘ |
| | 83° C. | >97° C. |
| HR2 peptide binding | Yes | Yes |
| K on/K off/Kd | | 4.3 × 10⁵/ |
| | | 3.3 × 10^{-3/} |
| | | 7.7 nM |
| Antiviral activity | | |
| against HIV IIIB | | |
| (μg/ml) | | |
| IC50 | 4.69 | 0.73 |
| IC90 | 23.24 | 3.03 |

*as determined by crystallography

C. This example illustrates a synthetic peptide provided with the present invention which, in addition to having amino acid substitutions in the “g” and “c” positions of the hydrophobic domain of the HR1 region of HIV-1 gp41, comprises an amino acid substitution in the “a” position of at least one of the heptads of the plurality of heptads comprising the peptide. Illustrating this example, a synthetic peptide having the amino acid sequence of SEQ ID NO:40 comprises the same amino acid sequence of the peptide having the amino acid sequence of SEQ ID NO:32, except, however, that a substitution was made in amino acid residue position corresponding to residue 48 (e.g., in the “a” position of at least one heptad in a plurality of heptads) of SEQ ID NO:32; and additionally, an aspartic acid and glutamine were added to the C-terminus of the peptide. Thus, the synthetic peptide having an amino acid sequence of SEQ ID NO:40 comprises a total length of 51 amino acid residues, with 6 complete heptads and 3 leucine zipper-like motifs. Such illustrative synthetic peptide (having the amino acid sequence of SEQ ID NO:40) was compared to a peptide derived from that native sequence of the HR1 region without any amino acid substitutions (SEQ ID NO:23) for oligomerization state, helicity, stability, antiviral activity, and binding to HR2 peptide, using methods as previously described in more detail in Example 1 herein. The comparisons of this illustrative synthetic peptide provided with the present invention to an HR1 peptide of native sequence is illustrated in Table 4.

TABLE 4
Biophysical and Antiviral Parameters

| | SEQ ID | SEQ ID |
|---|---|---|
| Parameter | NO:23 | NO:40 |
| Oligomerization | Aggregates | Trimer* |
| Helicity (at 10 μM) | 83% | 53% |
| Stability (at 10 μM) | Tₘ | Tₘ |
| | 83° C. | 62° C. |
| HR2 peptide | Yes | Yes |
| binding | | |
| Antiviral activity | | |
| against HIV IIIB | | |
| (μg/ml) | | |
| IC50 | 4.69 | 0.41 |
| IC90 | 23.24 | 1.84 |

*with only a small fraction (e.g., about 1 to about 10%) of higher order oligomer (e.g., hexamer) and therefore is considered to self assemble into trimers.

Also, one skilled in the art would appreciate from the data presented in Tables 2, 3, and 4 that the synthetic peptides have utility in a method for identifying or producing a compound or drug according to the present invention. More particularly, the synthetic peptide can self-assemble into trimers, thereby presenting a structure like that of the native trimeric core of the HR1 region of HIV gp41 to which can bind molecules (compounds and/or drugs) having binding specificity for such structure. Since the trimers employed with the present invention retain the ability to complex with HR2 peptide, provided is the means to evaluate the ability of a molecule to inhibit the binding interaction between the HR1 and HR2 regions of HIV gp41 (e.g., by contacting trimer, in the method according to the present invention, in the presence of HR2 peptide, with the molecule). Thus, a method of the present invention utilizing such trimers in vitro provide a means by which compounds can be identified or drugs produced which can inhibit/prevent six helix bundle formation between the trimer formed by the HR1 region of gp41 and trimer formed form the HR2 region of gp41 in forming a six-helix bundle in vivo. Further, as illustrated in FIG. 4, trimers formed from a synthetic peptide having the amino acid sequence of SEQ ID NO:36 also exhibit a cooperative transition at a narrow concentration range, beyond which no significant change is observed. Hence, a method according to the present invention, in which the the synthetic peptides (and trimers formed therefrom) are employed, represents a significant improvement and provides a significant advantage in identifying or producing a molecule that can inhibit the binding between HR1 and HR2 regions of HIV gp41.

D. In this example, highlighted is the importance in a synthetic peptide provided with the present invention to have amino acid substitutions in the “g” and “c” positions of the hydrophobic domain of the HR1 region of HIV-1 gp41. For comparative purposes, the peptide having the amino acid sequence of SEQ ID NO:41 has the same “a” and “d” substitutions as the synthetic peptide having the amino acid sequence of SEQ ID NO:35, but lacks the substitutions in the “g” position and the substitution in the “c” position of the hydrophobic domain. Similarly, the peptide having the amino acid sequence of SEQ ID NO:42 has the same “d” substitutions as the synthetic peptide having the amino acid sequence of SEQ ID NO:36, but lacks the substitution in the “g” position and the substitution in the “c” position of the hydrophobic domain. In both cases, as illustrated in Table 5, without the substitutions in the “g” and “c” positions of the hydrophobic domain, the resultant peptides demonstrate aggregate species in solution (e.g., oligomeric state other than predominately trimers), decrease in helicity, and decrease in antiviral activity, as compared to SEQ ID NO:35 and SEQ ID NO:36 (see Tables 2 and 3, respectively). The peptide having the amino acid sequence of SEQ ID NO:42 even has less antiviral activity than SEQ ID NO:23, the peptide derived from the native sequence of HIV-1 gp41 HR1. Thus, it can be concluded that substitutions in the “g” position and “c” position of the hydrophobic domain play a key role in the unexpected results with respect to biophysical and antiviral parameters of synthetic peptides used in the present invention, and trimers formed therefrom.

TABLE 5
Biophysical and Antiviral Parameters

| | SEQ ID | SEQ ID | SEQ ID |
|---|---|---|---|
| Parameter | NO:23 | NO:41 | NO:42 |
| Oligomerization | Aggregates | Soluble | Soluble |
| | | Aggregates | Aggregates |
| Helicity (at 10 μM) | 83% | 73% | 85% |
| Stability (at 10 μM) | Tₘ | Tₘ | Tₘ |
| | 83° C. | >97° C. | >97° C. |
| HR2 peptide binding | Yes | Yes | Not |
| | | | determined |
| | | | (precipitates) |
| Antiviral activity against | | | |
| HIV IIIB (μg/ml) | | | |
| IC50 | 4.69 | 3.892 | 13.605 |
| IC90 | 23.24 | 14.53 | 33.56 |

### EXAMPLE 3

In another embodiment, a synthetic peptide provided with the present invention comprises an amino acid substitution in either or both the C-terminal “e” position and “f” position in the hydrophobic domain of the HR1 region of the native sequence of HIV-1 gp41. The amino acid substitution in the C-terminal “e” position corresponds to amino acid residue position residue 35, and the amino acid substitution in the C-terminal “f” position corresponds to amino acid residue position residue 36, of SEQ ID NO:1 or polymorphisms thereof. Preferably, the amino acid substitution is with a hydrophobic amino acid other than valine (with respect to the “e” position) and tryptophan (with respect to the “f” position), more preferably with a hydrophobic amino acid with uncharged, nonpolar head groups (e.g., alanine and the like), and more preferably with alanine. More particularly, the HR1 region comprises a cluster of hydrophobic amino acids comprising the “hydrophobic domain”, wherein an amino acid substitution for one or more of L (leucine), V (valine), and W (tryptophan) in the hydrophobic domain disrupts the cluster sufficiently for synthetic peptide to be able to preferably self-assemble into trimers rather than some predominate oligomeric form other than trimers.

Synthetic peptides according to this embodiment of the present invention are exemplified by SEQ ID NOs:29, 30, and 43 which comprise residues 15 to 55 of SEQ ID NO:1 (and hence, comprise 41 amino acids), and additionally have the amino acid substitutions as described herein. These peptides were synthesized using the methods outlined in Example 1. A synthetic peptide having the amino acid sequence of SEQ ID NO:29 is comprised of 5 contiguous, complete heptads, and comprises the same sequence as a peptide having the amino acid sequence of SEQ ID NO:27 (native sequence from HIV-1 gp41 HR1), except that there is an amino substitution in the C-terminal “e” position of the hydrophobic domain (e.g., substitution of valine with alanine). This synthetic peptide (SEQ ID NO:29) illustrates a synthetic peptide comprising a substitution in the C-terminal “e” position in the hydrophobic domain. A synthetic peptide having the amino acid sequence of SEQ ID NO:30 is comprised of 5 contiguous, complete heptads, and comprises the same sequence as the peptide having the amino acid sequence of SEQ ID NO:27, except that there is an amino substitution in the C-terminal “f” position of the hydrophobic domain, whereby the tryptophan is substituted with an alanine. This synthetic peptide (SEQ ID NO:30) illustrates a synthetic peptide comprising a substitution in the C-terminal “f” position in the hydrophobic domain. A synthetic peptide having the amino acid sequence of SEQ ID NO:43 also comprises five contiguous, complete heptads, and comprises the same sequence as peptide having the amino acid sequence of SEQ ID NO:27, except that there is an amino acid substitution in the C-terminal “f” position of the hydrophobic domain, where the tryptophan is substituted with a phenylalanine. This synthetic peptide (SEQ ID NO:43) is another illustration of a synthetic peptide comprising a substitution in the C-terminal “f” position in the hydrophobic domain.

A further example of the present invention is a synthetic peptide corresponding to amino acid residues 5 to 53 of SEQ ID NO:1 (having an amino acid sequence of SEQ ID NO:31), which possesses the same amino acid sequence as the HR1 peptide having the amino acid sequence of SEQ ID NO:23 (native sequence from HIV-1 gp41 HR1) except that a phenylalanine is substituted for the tryptophan in the C-terminal “f” position of the hydrophobic domain, and an arginine is substituted for a lysine at an amino acid residue corresponding to the “b” position of the heptad adjoining (the C-terminus of) the hydrophobic domain (e.g., corresponding to amino acid residue position 39 of SEQ ID NO:1). The synthetic peptide having the amino acid sequence of SEQ ID NO:31 comprises six complete, contiguous heptads.

Synthetic peptides having the amino acid sequences of SEQ ID NOs: 29, 30, 31 and 43, exemplary of an embodiment of synthetic peptides according to the present invention, were compared to HR1 peptide (SEQ ID NO:27) for oligomerization state, helicity, stability, and binding to HR2 peptide (SEQ ID NO:33) using methods as previously described in more detail in Example 1 herein. The comparison of synthetic peptides having the amino acid sequences of SEQ ID NOs:29, 30, 31 and 43 to HR1 peptide (SEQ ID NO:27) is illustrated in Table 6.

It is an unexpected result that an amino acid substitution in the either of the C-terminal “e” position and/or in the C-terminal “f” position of the hydrophobic domain, confers the oligomeric state of synthetic peptide to that comprising predominately a trimer in solution (as can be concluded from the data presented in Table 6). More particularly, amino acid substitutions in amino acid residues neighboring (i.e., at the “g” position in the same or adjacent heptad) the C-terminal “e” and “f” positions of the hydrophobic domain failed to switch the oligomeric state to self-assembly into trimers (see, e.g., peptides having the amino acid sequences of SEQ ID NOs: 44-46; and each of which self-assembles into tetramers in solution). Also as shown in Table 6, an amino acid substitution in the either of the C-terminal “e” position and/or in the C-terminal “f” position of the hydrophobic domain can result in a significant increase in helicity for the synthetic peptide, as well as an increase in antiviral activity (e.g., increase in potency; at least a 3 fold increase in potency) as compared to an HR1 peptide of the native sequence (without substutions).

TABLE 6
Biophysical and antiviral Parameters

| | SEQ ID | SEQ ID | SEQ ID | SEQ ID | SEQ ID |
|---|---|---|---|---|---|
| Parameter | NO:27 | NO:29 | NO:30 | NO:31 | NO:43 |
| Oligomerization | Tetramer | Trimer* | Trimer* | Trimer** | Trimer** |
| Helicity | 72% | 78% | 84% | 86% | 94% |
| (10 μM, 25° C.) | | | | | |
| Stability | Tₘ 76° C. | Tₘ 65° C. | Tₘ 77° C. | Tₘ 70° C. | Tₘ 74° C. |
| (at 10 μM) | | | | | |
| HR2 peptide | Yes | Yes | Not | Yes | Yes |
| Binding | | | determined | | |
| K on/K off/Kd | | | (precipitates) | | |
| Antiviral activity | | | | | |
| Vs. HIV IIIB: | | | | | |
| (μg/ml) | | | | | |
| IC50 | 4.69 | 2.81 | 0.37 | 0.93 | 1.34 |
| IC90 | 23.24 | 5.86 | 1.00 | 3.06 | 3.81 |

*consistent with weight-averaged MW analysis
**with only a small fraction (e.g., about 1% to about 10%) of higher order oligomer (e.g., hexamer) and therefore, is considered to self assemble predominately into trimers.

Also, one skilled in the art would appreciate from the data presented in Table 6 that trimers formed from synthetic peptide have utility in a method for identifying or producing a compound or drug according to the present invention. More particularly, the synthetic peptide can self-assemble into trimers, thereby presenting a structure like that of the native trimeric core of the HR1 region of HIV gp41 to which can bind molecules (compounds and/or drugs) having binding specificity for such structure. Since the trimers employed with the present invention retain the ability to complex with HR2 peptide, provided is the means to evaluate the ability of a molecule to inhibit the binding interaction between the HR1 and HR2 regions of HIV gp41 (e.g., by contacting trimer, in the method according to the present invention, in the presence of HR2 peptide, with the molecule) in vivo.

### EXAMPLE 4

In an embodiment of a method according to the present invention for identifying or producing a molecule (e.g., drug or compound) that can inhibit transmission of HIV to a target cell, preferably identified or produced is a molecule that can inhibit the binding between HR1 and HR2 regions of HIV gp41. In this regard, generally a method according to the present invention employs trimers, formed from synthetic peptide, which present a structure like that of the trimeric core of the HR1 region of gp41 to which molecules can bind thereby inhibiting six-helix bundle formation between the HR1 region and HR2 region of gp41 in the fusion process of HIV in vivo. In that regard, typical concentrations of synthetic peptide comprising trimers used in this method are generally in the nM range, but other concentrations may be used. However, an exception is synthetic peptide comprising trimers provided with the present invention, SEQ ID NO:32, which may be in a monomer and trimer equilibrium at low concentrations (e.g., less than 50 nM) in solution. Hence, use of such synthetic peptide at such concentrations in the methods described herein, also provided is a means for identifying or producing a molecule that can inhibit or disrupt HR1 trimer formation in vitro, thus having applications for inhibiting HR1 trimer formation in vivo for inhibiting the gp41-mediated fusion process of HIV. As apparent to one skilled in the art, various approaches may be pursued in identifying or producing a molecule that can inhibit transmission of HIV to a target cell. In one example, a solid phase-based assay may be utilized to detect the ability of the molecule to inhibit complex formation (e.g., to the assay system is added the molecule prior to or in conjunction with complex formation) or disrupt binding (e.g., added to the assay system is the molecule after substantial complex formation has been completed) between either trimers (comprised of synthetic peptide) or synthetic peptide (e.g., SEQ ID NO:32) employed with the present invention, and HR2 peptide. For ease of description, trimer or synthetic peptide provided with the present invention is referred to as a “binding partner” for a substrate comprising HR2 peptide referred to as another “binding partner”. Thus, for example, a solid phase-based assay would include the attachment of either of the binding partners to a solid surface. Illustrative, but nonlimiting, examples of solid surfaces used for such purposes include: agarose or plastic or synthetic beads; microtiter plate wells; petri dishes; synthetic membranes (e.g., composed of nylon, or nitrocellulose, or the like); microarrays; microfluidic devices (e.g., used for high throughput), “protein” chips, and the like. A macromolecular carrier may be used to facilitate attachment of a binding partner to a solid surface (e.g., polylysine is known by those skilled in the art for such purpose).

In such an assay system, either of the binding partners may be immobilized onto a solid surface. The remaining binding partner, which is not immobilized, may be labeled, either indirectly or directly, with a detectable moiety. Thus, for example, if trimers are immobilized to the solid surface, the HR2 peptide may be labeled with a detectable moiety. Alternatively, the molecule being added to the assay system may be labeled, either directly or indirectly, with a detectable moiety and the binding partner may be unlabeled, or labeled with a detectable moiety that can be detectably distinguished from the label used in conjunction with the molecule (e.g. spectrally distinguishable detectable moieties used in combination are well known in the art). In yet another alternative, both of the binding partners may be labeled with detectable moiety, particularly if each binding partner is labeled a detectable moiety which is spectrally distinguishable from the detectable moiety used to label the other binding partner. In another example, the molecule may be the immobilized component in the assay system, and added to the immobilized component are the trimers and HR2 peptide. In either case, the component (molecule or binding partner) to be immobilized may be immobilized by non-covalent or covalent attachments known in the art (e.g., simply by coating the solid surface with a solution of the component and drying; or by a specific affinity ligand or chemical bonding process). Detectable moieties are known in the art to include, but are not limited to, enzymes (e.g., peroxidase, alkaline phosphatase, etc.), radioisotopes, haptens (e.g., biotin, avidin, etc.), chromophores, luminescers, fluorescent molecules, and fluorescent nanocrystals, as known to those skilled in the art of assay systems. Most detectable moieties will allow detection in a choice of either or both quantitatively or qualitatively, and depending on the type of assay system utilized.

In a solid phase-based assay system, generally between reaction steps there may be a washing step to remove unbound reactants from the assay system (“heterogenous solid phase assay”). However, a washing step may not be required (“homogenous solid phase assay”), depending on such factors as the reactants, their concentration, the order in which they are reacted, and the type of assay system utilized. For example of a heterogenous solid phase assay in performing the method according to the present invention, trimers formed from synthetic peptide were immobilized in wells of a microtiter plate. Molecule and biotinylated H2 peptide were added to the reaction in buffer for a sufficient time to contact the immobilized trimers. The plate was washed to remove components which were not complexed to the trimers. Binding of biotinylated HR2 peptide was then enzymatically detected by a second reaction step in which was added streptavidin-labeled horseradish peroxidase (followed by a washing step to remove unbound streptavidin-HRP). Thus, a control well may contain just trimers employed with the present invention contacted with labeled-HR2 peptide, to compare with wells in which molecule was additionally added, as a reference value to detect (and quantitate, if desired) inhibition of complexing between HR1 and HR2 caused by the molecule. As apparent to one skilled in the art, the detection of the complexes which are bound to the solid surface via an immobilized component can be detected by measuring the labeled reactant (binding partner and/or molecule, depending on the assay type) using appropriate detection means for the detectable moiety used to label the reactant.

In another example of the method according to the present invention, the assay system may comprise a liquid phase-based assay system wherein the binding partners and the molecule are contacted together in solution. The complexes formed may then be detected directly in the assay system or by first separating them from unreacted (non-complexed) components), depending on the type of assay system used, the detectable moiety, the detection means for the detectable moiety, and the like. In either type of system (solid phase-based or liquid phase-based), the order of the reactants can be varied to obtain different information concerning the biological activity of the molecules being identified or produced. For example, to assess the ability of the molecule to inhibit complex formation between the binding partners, then desirably the molecule should be contacted with the binding partners (synthetic peptide comprising trimers and the HR2 peptide) prior to or simultaneous with contacting the two binding partners (synthetic peptide comprising trimers and the HR2 peptide). In another example, to assess the ability of the molecule to disrupt complexes formed between the binding partners, then desirably the molecule should be contacted with the binding partners (synthetic peptide comprising trimers and the HR2 peptide) after the binding partners have been contacted together and after a time sufficient for complexing to occur between the binding partners.

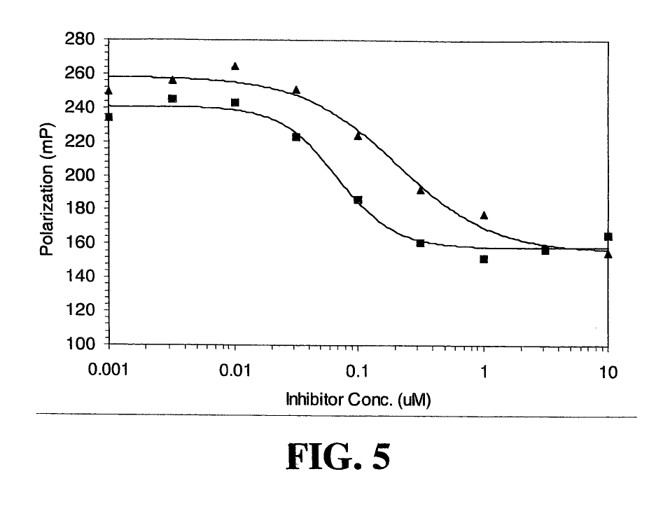

In an example of a homogenous assay carried out as a liquid phase-based assay system, a fluorescence polarization assay was used in taking advantage of the high affinity interaction between HR1 and HR2 regions. Into reaction wells of a high throughput plate (>300 reaction wells) was added 20 μl of a solution containing a desired concentration of molecule to be assessed for its ability to bind to trimers employed with the present invention (e.g., formed from synthetic peptide) and either inhibit or disrupt HR1-HR2 binding interactions (“complexing between HR1 and HR2″). Reference standards (e.g., predetermined amounts of a known inhibitor of HR1-HR2 binding interactions, or no inhibitor) are added to other reaction wells (not containing the molecule to be assessed) in various concentrations to determine, for example, values reflecting one or more of no inhibition or disruption, maximum binding, 50% inhibition or disruption, and maximum inhibition or disruption. To each reaction well was then added 10 μl of a solution containing a desired concentration of trimers (formed from synthetic peptide employed with the present invention). After a sufficient time for the trimers to contact molecule (or reference standard in reaction wells containing reference standard but not molecule) (e.g., 15 minute incubation period was used, but this may vary depending on the type of assay and assay components),10 μl of a solution containing a desired concentration of HR2 peptide labeled with a detectable moiety was added to the reaction wells. With regards to the various solutions of reactants for this assay, the components of the respective solution, its volume, and concentration of reactant (molecule or trimers, or labeled HR2 peptide) may be varied depending on the nature of the reactant, size of sample well, type of detectable moiety used to label the HR2 peptide, and other factors. In this example, the detectable moiety comprised a fluorescent dye (e.g., rhodamine and/or fluorescein) and the assay is then allowed to proceed for a sufficient period of time for labeled HR2 peptide to bind (or compete with molecule for binding) to the trimers. Fluorescence intensity of polarized light was then measured with a detection system comprising a spectrophotometer, and the amount of polarized light was calculated using standard software in the art. In this example of the method according to the present invention, molecules assessed for ability to inhibit or disrupt complexing between trimers and a labeled HR2 peptide (e.g., SEQ ID NO:33) comprised either of two HR2 peptides. In that regard, HR2 peptide A (SEQ ID NO:33) and HR2 peptide B (SEQ ID NO:49) were individually added in various concentrations up to 1 nM in buffer to fluorescein-labeled HR2 peptide ( concentration of 0.2 nM), and the trimers employed with the present invention (SEQ ID NO:36; concentration of 50 nM). As shown in FIG. 5, it was determined from the method according to the present invention that both molecules (HR2 peptide A, FIG. 5 ”□”; and HR2 peptide B, FIG. 5 “▴”) are compounds that inhibit binding interactions (e.g., complexing) between HR1 and HR2 regions (e.g., as compared to the amount of complex formation detected in the absence of the molecules), and that the concentration to inhibit complexing, between the trimers and labeled HR2 peptide, by 50% (IC₅₀) was 68 nM for HR2 peptide A and 200 nM for HR2 peptide B. It is also important to note that each of HR2 peptide A and HR2 peptide B has shown antiviral activity against HIV-1 in the in vitro infectivity assay (HR2 peptide A: IC₅₀ of 0.002 μg/ml, IC₉₀ of 0.072 μg/ml; HR2 peptide B: IC₅₀ of 0.010 μg/ml, IC₉₀ of 0.078 μg/ml), and has been mixed with a pharmaceutically acceptable carrier. Hence, the method of the present invention may be used for identifying or producing a molecule that can inhibit transmission of HIV to a target cell.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept; and thus, such modifications and/or adaptations are intended to be within the meaning and scope of the appended claims.

What is claimed is:

1. A method for identifying a compound that inhibits transmission of HIV to a target cell, the method comprising contacting synthetic peptide comprising trimers in the presence of a compound and with HR2 peptide under conditions and for a time sufficient to allow formation of a complex between the synthetic peptide comprising trimers and HR2 peptide in vitro; and detecting the amount of complex formed; wherein inhibition or reduction of complex formation in the presence of the compound, as compared to complex formation in the absence of the compound, is indicative of ability of the compound to inhibit transmission of HIV to a target cell; and
wherein synthetic peptide comprises an amino acid sequence derived from the HR1 region of HIV-1 gp41; wherein the HR1 region consists of native amino acid sequence shown as SEQ ID NO:1 or polymorphisms thereof; wherein the HR1 region from which the synthetic peptide is derived comprises a hydrophobic domain of amino acids corresponding to amino acid residues in positions 28 to 36 of SEQ ID NO:1 or polymorphisms thereof; wherein the amino acid residues comprising the hydrophobic domain correspond to heptad repeat positions “efgabcdef”; and wherein the amino acid sequence of the synthetic peptide further comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

2. The method according to claim 1, wherein the amino acid sequence of the synthetic peptide comprises one or more amino acid substitutions in the hydrophobic domain comprising either a substitution in the “c” position, or a substitution in both the “g” position and the “c” position, of the heptad repeat positions “efgabcdef”.

3. The method according to claim 2, wherein the synthetic peptide comprises an amino acid substitution additional to a substitution in either the “c” position or both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

4. The method according to claim 1, wherein the amino acid sequence of the synthetic peptide comprises one or more amino acid substitutions in the hydrophobic domain comprising the heptad repeat positions “efgabcdef” that are in a position of the heptad repeat positions selected from the group consisting of a C-terminal “e” position, a C-terminal “f” position, and a combination thereof.

5. The method according to claim 4, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of the “a” position, a “d” position, a “b” position, and a combination thereof.

6. The method according to claim 1, wherein the synthetic peptide further comprises a component selected from the group consisting of one or more reactive functionalities, a macromolecular carrier, a pharmaceutically acceptable carrier, an amino acid substitution comprising an addition of no less than one amino acid and no more than twenty amino acids to either or both of the amino terminus or carboxy terminus of the synthetic peptide, and a combination thereof.

7. The method according to claim 1, wherein the synthetic peptide is predominately in trimeric form.

8. The method according to claim 1, wherein the synthetic peptide is in a monomer-trimer equilibrium.

9. A method for producing a drug that inhibits transmission of HIV to a target cell, the method comprising contacting synthetic peptide comprising trimers in the presence of a compound and with HR2 peptide under conditions and for a time sufficient to allow formation of a complex between the synthetic peptide comprising trimers and HR2 peptide in vitro; and detecting the amount of complex formed; wherein inhibition or reduction of complex formation in the presence of the compound is indicative of ability of the compound to inhibit transmission of HIV to a target cell; and wherein the drug comprises the compound contacted with a pharmaceutically acceptable carrier in producing the drug; and
wherein synthetic peptide comprises an amino acid sequence derived from the HR1 region of HIV-1 gp41; wherein the HR1 region consists of native amino acid sequence shown as SEQ ID NO:1 or polymorphisms thereof; wherein the HR1 region from which the synthetic peptide is derived comprises a hydrophobic domain of amino acids corresponding to amino acid residues in positions 28 to 36 of SEQ ID NO:1 or polymorphisms thereof; wherein the amino acid residues comprising the hydrophobic domain correspond to heptad repeat positions “efgabcdef”; and wherein the amino acid sequence of the synthetic peptide further comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

10. The method according to claim 9, wherein the amino acid sequence of the synthetic peptide comprises one or more amino acid substitutions in the hydrophobic domain comprising either a substitution in the “c” position, or a substitution in both the “g” position and the “c” position, of the heptad repeat positions “efgabcdef”.

11. The method according to claim 10, wherein the synthetic peptide comprises an amino acid substitution additional to a substitution in either the “c” position or both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

12. The method according to claim 9, wherein the amino acid sequence of the synthetic peptide comprises one or more amino acid substitutions in the hydrophobic domain comprising the heptad repeat positions “efgabcdef” that are in a position of the heptad repeat positions selected from the group consisting of a C-terminal “e” position, a C-terminal “f” position, and a combination thereof.

13. The method according to claim 12, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of the “a” position, a “d” position, a “b” position, and a combination thereof.

14. The method according to claim 9, wherein the synthetic peptide further comprising a component selected from the group consisting of one or more reactive functionalities, a macromolecular carrier, a pharmaceutically acceptable carrier, an amino acid substitution comprising an addition of no less than one amino acid and no more than twenty amino acids to either or both of the amino terminus or carboxy terminus of the synthetic peptide, and a combination thereof.

15. The method according to claim 9, wherein the synthetic peptide is predominately in trimeric form.

16. The method according to claim 9, wherein the synthetic peptide is in a monomer-trimer equilibrium.

17. In a method for identifying or producing a molecule that can inhibit the binding between HR1 and HR2 regions of HIV gp41, the improvement which comprises: use of a trimer as a binding partner with HR2 peptide in detecting in vitro the ability of the molecule to bind to an HR (heptad repeat) region of HIV gp41;
wherein the trimer is comprised of synthetic peptide comprising an amino acid sequence derived from the HR1 region of HIV-1 gp41; wherein the HR1 region consists of native amino acid sequence shown as SEQ ID NO:1 or polymorphisms thereof;
wherein the HR1 region from which the synthetic peptide is derived comprises a hydrophobic domain of amino acids corresponding to amino acid residues in positions 28 to 36 of SEQ ID NO:1 or polymorphisms thereof; wherein the amino acid residues comprising the hydrophobic domain correspond to heptad repeat positions “efgabcdef”;
and wherein the amino acid sequence of the synthetic peptide further comprises one or more amino acid substitutions in the heptad repeat positions “efgabcdef” comprising the hydrophobic domain, as compared to the native amino acid sequence of the HR1 region, which enables synthetic peptide to self-assemble in solution into trimers.

18. The method according to claim 17, wherein the amino acid sequence of the synthetic peptide comprises one or more amino acid substitutions in the hydrophobic domain comprising either a substitution in the “c” position, or a substitution in both the “g” position and the “c” position, of the heptad repeat positions “efgabcdef”.

19. The method according to claim 18, wherein the synthetic peptide comprises an amino acid substitution additional to a substitution in either the “c” position or both the “g” position and “c” position, wherein the additional amino acid substitution is in one or more amino acid positions of one or more heptads of the synthetic peptide, and wherein the one or more amino acid positions is selected from the group consisting of an “a” position, a “d” position, a “b” position, and a combination thereof.

20. The method according to claim 17, wherein the amino acid sequence of the synthetic peptide comprises one or more amino acid substitutions in the hydrophobic domain comprising the heptad repeat positions “efgabcdef” that are in a position of the heptad repeat positions selected from the group consisting of a C-terminal “e” position, a C-terminal “f” position, and a combination thereof.

21. The method according to claim 20, wherein the synthetic peptide comprises an amino acid substitution additional to the substitution in one or more of the “e” position and the “f” position, wherein the additional amino acid substitution is in one or more amino acid positions of one or more heptads of the synthetic peptide, and wherein the one or more amino acid positions is selected from the group consisting of the “a” position, a “d” position, a “b” position, and a combination thereof.

22. The method according to claim 17, wherein the synthetic peptide further comprising a component selected from the group consisting of one or more reactive functionalities, a macromolecular carrier, a pharmaceutically acceptable carrier, an amino acid substitution comprising an addition of no less than one amino acid and no more than twenty amino acids to either or both of the amino terminus or carboxy terminus of the synthetic peptide, and a combination thereof.


SEQUENCE LISTING (48 sequences)

SEQ ID NO: 1 - protein, 59 aa, Human immunodeficiency virus
TLTVQARQLLSGIVQQQNNLLRAIEAQQHLLQLTVWGIKQLQARILAVERYLKDQLLGI

SEQ ID NO: 2 - protein, 38 aa, Artificial
  synthesized
NNLLRAIEAQQHLLQLTVWGIKQLQARILAVERYLKDQ

SEQ ID NO: 3 - protein, 43 aa, Artificial
  synthesized
GSTMGARSMTLTVQARQLLSGIVQQQNNLLRAIEAQQHLLQLT

SEQ ID NO: 4 - protein, 54 aa, Artificial
  synthesized
GSTMGARSMTLTVQARQLLSGIVQQQNNLLRAIEAQQHLLQLTVWGIKQLQARI

SEQ ID NO: 5 - protein, 36 aa, Artificial
  synthesized
GARSMTLTVQARQLLSGIVQQQNNLLRAIEAQQHLL

SEQ ID NO: 6 - protein, 38 aa, Artificial
  synthesized
GARSMTLTVQARQLLSGIVQQQNNLLRAIEAQQHLLQL

SEQ ID NO: 7 - protein, 40 aa, Artificial
  synthesized
GARSMTLTVQARQLLSGIVQQQNNLLRAIEAQQHLLQLTV

SEQ ID NO: 8 - protein, 50 aa, Artificial
  synthesized
GARSMTLTVQARQLLSGIVQQQNNLLRAIEAQQHLLQLTVWGIKQLQARI

SEQ ID NO: 9 - protein, 36 aa, Artificial
  synthesized
ARSMTLTVQARQLLSGIVQQQNNLLRAIEAQQHLLQ

SEQ ID NO: 10 - protein, 36 aa, Artificial
  synthesized
RSMTLTVQARQLLSGIVQQQNNLLRAIEAQQHLLQL

SEQ ID NO: 11 - protein, 36 aa, Artificial
  synthesized
SMTLTVQARQLLSGIVQQQNNLLRAIEAQQHLLQLT

SEQ ID NO: 12 - protein, 35 aa, Artificial
  synthesized
MTLTVQARQLLSGIVQQQNNLLRAIEAQQHLLQLT

SEQ ID NO: 13 - protein, 36 aa, Artificial
  synthesized
MTLTVQARQLLSGIVQQQNNLLRAIEAQQHLLQLTV

SEQ ID NO: 14 - protein, 34 aa, Artificial
  synthesized
TLTVQARQLLSGIVQQQNNLLRAIEAQQHLLQLT

SEQ ID NO: 15 - protein, 35 aa, Artificial
  synthesized
TLTVQARQLLSGIVQQQNNLLRAIEAQQHLLQLTV

SEQ ID NO: 16 - protein, 36 aa, Artificial
  synthesized
TLTVQARQLLSGIVQQQNNLLRAIEAQQHLLQLTVW

SEQ ID NO: 17 - protein, 37 aa, Artificial
  synthesized
TLTVQARQLLSGIVQQQNNLLRAIEAQQHLLQLTVWG

SEQ ID NO: 18 - protein, 38 aa, Artificial
  synthesized
TLTVQARQLLSGIVQQQNNLLRAIEAQQHLLQLTVWGI

SEQ ID NO: 19 - protein, 44 aa, Artificial
  synthesized
TLTVQARQLLSGIVQQQNNLLRAIEAQQHLLQLTVWGIKQLQAR

SEQ ID NO: 20 - protein, 36 aa, Artificial
  synthesized
LTVQARQLLSGIVQQQNNLLRAIEAQQHLLQLTVWG

SEQ ID NO: 21 - protein, 42 aa, Artificial
  synthesized
QARQLLSGIVQQQNNLLRAIEAQQHLLQLTVWGIKQLQARIL

SEQ ID NO: 22 - protein, 47 aa, Artificial
  synthesized
QARQLLSGIVQQQNNLLRAIEAQQHLLQLTVWGIKQLQARILAVERY

SEQ ID NO: 23 - protein, 49 aa, Artificial
  synthesized
QARQLLSGIVQQQNNLLRAIEAQQHLLQLTVWGIKQLQARILAVERYLK

SEQ ID NO: 24 - protein, 51 aa, Artificial
  synthesized
QARQLLSGIVQQQNNLLRAIEAQQHLLQLTVWGIKQLQARILAVERYLKDQ

SEQ ID NO: 25 - protein, 36 aa, Artificial
  synthesized
SGIVQQQNNLLRAIEAQQHLLQLTVWGIKQLQARIL

SEQ ID NO: 26 - protein, 45 aa, Artificial
  synthesized
SGIVQQQNNLLRAIEAQQHLLQLTVWGIKQLQARILAVERYLKDQ

SEQ ID NO: 27 - protein, 41 aa, Artificial
  synthesized
QQQNNLLRAIEAQQHLLQLTVWGIKQLQARILAVERYLKDQ

SEQ ID NO: 28 - protein, 34 aa, Artificial
  synthesized
RAIEAQQHLLQLTVWGIKQLQARILAVERYLKDQ

SEQ ID NO: 29 - protein, 41 aa, Artificial
  synthesized
QQQNNLLRAIEAQQHLLQLTAWGIKQLQARILAVERYLKDQ

SEQ ID NO: 30 - protein, 41 aa, Artificial
  synthesized
QQQNNLLRAIEAQQHLLQLTVAGIKQLQARILAVERYLKDQ

SEQ ID NO: 31 - protein, 49 aa, Artificial
  synthesized
QARQLLSGIVQQQNNLLRAIEAQQHLLQLTVFGIRQLQARILAVERYLK

SEQ ID NO: 32 - protein, 49 aa, Artificial
  synthesized
QARQLLSGIVQQQNNLLRAIEAQQHALQATVWGIKQLQARILAVERYLK

SEQ ID NO: 33 - protein, 36 aa, Artificial
  synthesized
WMEWDREINNYTSLIHSLIEESQNQQEKNEQELLEL

SEQ ID NO: 34 - protein, 51 aa, Artificial
  synthesized
QARQLLSGIVQQQNNLLRAIEAQQHALQATVWGIKQLQARILAVERYLKDQ

SEQ ID NO: 35 - protein, 49 aa, Artificial
  synthesized
QARQLVSGLVQQQNNILRALEATQHAVQALVWGVKQLQARVLALERYIK

SEQ ID NO: 36 - protein, 49 aa, Artificial
  synthesized
QIRQLLSGIVQQQNNLLRAIEAIQHALQAIVWGIKQLQARILAVERYLK

SEQ ID NO: 37 - protein, 49 aa, Artificial
  synthesized
QARQLVSGLVQQQNNILRALEATQHAVQALVWGVRQLQARVLALERYIK

SEQ ID NO: 38 - protein, 51 aa, Artificial
  synthesized
QARQLLSGIVQQQNNLLRAIEATQHAVQALVWGVKQLQARVLALERYIKDQ

SEQ ID NO: 39 - protein, 51 aa, Artificial
  synthesized
QARQLVSGLVQQQNNILRALEAQQHALQATVWGIKQLQARVLALERYIKDQ

SEQ ID NO: 40 - protein, 51 aa, Artificial
  synthesized
QARQLLSGIVQQQNNLLRAIEAQQHALQATVWGVKQLQARILAVERYLKDQ

SEQ ID NO: 41 - protein, 49 aa, Artificial
  synthesized
QARQLVSGLVQQQNNILRALEATQHLVQLLVWGVKQLQARVLALERYIK

SEQ ID NO: 42 - protein, 49 aa, Artificial
  synthesized
QIRQLLSGIVQQQNNLLRAIEAIQHLLQLIVWGIKQLQARILAVERYLK

SEQ ID NO: 43 - protein, 41 aa, Artificial
  synthesized
QQQNNLLRAIEAQQHLLQLTVFGIKQLQARILAVERYLKDQ

SEQ ID NO: 44 - protein, 41 aa, Artificial
  synthesized
QQQNNLLRAIEAQQHLLQLTVWGIAQLQARILAVERYLKDQ

SEQ ID NO: 45 - protein, 41 aa, Artificial
  synthesized
QQQNNLLRAIEAQQHLLQLTVWGIKQLAARILAVERYLKDQ

SEQ ID NO: 46 - protein, 41 aa, Artificial
  synthesized
QQQNNLLRAIEAQQHALQLTVWGIKQLQARILAVERYLKDQ

SEQ ID NO: 47 - protein, 51 aa, Artificial
  synthesized
QARQLLSGIVQQQNNLLRAIEAQQHLLQATVWGIKQLQARILAVERYLKDQ

SEQ ID NO: 48 - protein, 41 aa, Artificial
  synthesized
QQQNNLLRAIEAQQHLLQATVWGIKQLQARILAVERYLKDQ